(19) United States
(12) Patent Application Publication
(10) Pub. No.: US 2011/0256576 A1
(43) Pub. Date: Oct. 20, 2011

(54) RECOVERY AND DETECTION OF MICROORGANISMS FROM MIXED CELLULOSE ESTER FILTRATION SUPPORTS BY SEQUENTIAL TREATMENT WITH METHANOL AND ACETONE

(75) Inventors: Luc Bissonnette, Quebec (CA); Andrée F. Maheux, Val-Belair (CA); Michel G. Bergeron, Quebec (CA); Maurice Boissinot, Saint-Augustin-de-Desmaures (CA); Jean-Luc Bernier, Quebec (CA)

(73) Assignee: Universite Laval, Quebec (CA)

(21) Appl. No.: 13/122,929

(22) PCT Filed: Oct. 14, 2009
(86) PCT No.: PCT/CA09/01465

§ 371 Date: Jul. 4, 2011

Related U.S. Application Data
(60) Provisional application No. 61/136,903, filed on Oct. 14, 2008.

(51) Int. Cl. C12N 1/00; C12N 11/00; C12Q 1/02; C12N 1/10; C12N 1/14; C12N 7/02; C12N 1/20

(52) U.S. Cl. 435/29; 435/239; 435/243; 435/174; 435/252.1; 435/258.1; 435/254.1; 435/252.5; 435/252.8

(57) ABSTRACT

The present invention is directed to the recovery of bacteria and microparasites, particularly Cryptosporidium and Giardia, from water samples by filtration through a mixed cellulose ester membrane, partial dissolution of said membrane with methanol followed by completion in the presence of acetone, and purification and concentration using glass beads as a secondary confinement matrix.

### FIELD OF THE INVENTION

The present invention relates to the recovery and/or concentration of cells or microorganisms from filtration supports. The present invention also relates to the detection of cells or microorganisms that are recovered and/or concentrated using the method of the present invention.

### BACKGROUND OF THE INVENTION

Membrane filtration is an approach widely used to remove, trap, concentrate, and/or purify chemical and/or biological components (e.g. ions, molecules, macromolecules, particles, viruses, microorganisms, cells, etc.) of various gaseous or liquid matrices, such as air or water. In environmental microbiology, water production industry, agri-food industry or occupational health applications, membrane filtration is used, for example, to establish the safety of putatively contaminated water or air samples by determining the presence of index or pathogen microorganisms. In a general application of the method, exploited for the detection of microbial contaminants by classical microbiology procedures, a water sample is filtered through a filtration membrane and the whole filter is deposited on a solid culture medium allowing the growth and eventual detection of fecal contamination indicators after a defined time of incubation under suitable conditions (Eduard W. and Heederik D., Am. Ind. Hyg. Assoc. J. 59: 113-127, 1998; Rompré A. et al., J. Microbiol. Meth. 49: 31-54, 2002). Depending on the nature or the composition of the matrix to filter, the filtration membrane (filtration support) may be made of polyamide, polycarbonate, polyethersulfone, polyvinylidene fluoride, nylon, nitrocellulose, mixed cellulose esters, polypropylene, etc. Depending on the particular application, the filtration membrane may be use in a simple holder or folded, pleated, stacked, or oriented perpendicularly or tangentially against the flow, in more elaborate filtration cartridges, capsules, or devices (Eduard W. and Heederik D., Am. Ind. Hyg. Assoc. J. 59: 113-127, 1998; van Reis R. and Zydney A., Curr. Opin. Biotechnol. 12: 208-211, 2001; Heberer T. et al., Acta Hydrochim. Hydrobiol. 30: 24-33, 2002; Stetzenbach L. D. et al., Curr. Opin. Biotechnol. 15: 170-174, 2004; Levy R. V. and Jornitz M. W., Adv. Biochem. Engin. Biotechnol. 98: 1-26, 2006).

In some cases, for example where microbial particles (microorganisms) may be unculturable or difficult to grow in cultures, or more easily detectable by molecular-based methods, or simply requires concentration, microbial particles are concentrated from liquid samples by either sedimentation and/or flocculation methods, filtration and/or ultrafiltration on a flat membrane, deep filter or cartridge, centrifugation, and/or immunomagnetic separation. For air samples filtration, electrostatic precipitation, liquid impingement, or impaction are preferred concentration methods. Subsequently, particles or microorganisms may be released from the concentration receptacle (filtration support, membrane, cartridge, container, funnel, deep filter, etc.) before detection and/or enumeration methods are performed (Hsu B.-M. and C. Huang, J. Environ. Qual. 29: 1587-1593, 2000; Carey C. M. et al., Water Res. 38: 818-862, 2004; Stetzenbach L. D. et al., Curr. Opin. Biotechnol. 15: 170-174, 2004; Zarlenga D. S. and J. M. Trout, Vet. Parasitol. 126: 195-217, 2004).

However, the macromolecular components of the outer shell or surface may modify the overall charge and/or hydrophobicity of microbial particles, thereby altering their adhesive, electrostatic, or adsorptive interactions with various animate or inanimate surfaces as exemplified with bacterial endospores and exospores (Ahimou F. et al., J. Microbiol. Meth. 45: 119-126, 2001; Faille C. et al., Can. J. Microbiol. 48: 728-738, 2002), protozoan, parasites oocysts, cysts or spores (Drozd C. and J. Schwartzbrod, Appl. Environ. Microbiol. 62: 1227-1232, 1996; Butkus M A et al., Appl. Environ. Microbiol. 69: 3819-3825, 2003; Graczyk T. K. et al., Am. J. Trop. Med. Hyg. 68: 228-232, 2003; Carey C. M. et al., Water Res. 38: 818-862, 2004), fungal endospores or exospores (Slawecki R. A. et al., Appl. Environ. Microbiol. 68: 597-601, 2002; Linder M. B. et al., FEMS Microbiol. Rev. 29: 877-896, 2005), and viruses and bacteriophages (Lukasik J. et al., Appl. Environ. Microbiol. 66: 2914-2920, 2000). The release or dislodgement of microbial particles from the concentration receptacle or filtration support may be generally performed by physical means such as elution, backwashing, sonication, mechanical removal (scraping, vigorous mixing, etc.), and/or chemical removal, an/or cell lysis, and/or dissolution of the filtration membrane by chemical degradation of its physical integrity, with variable levels of efficiency (Aldom J. E. and A. H. Chagla, Lett. Appl. Microbiol. 20: 186-187, 1995, Ferguson C. et al., Can J. Microbiol. 50: 675-682, 2004; Burton N. C. et al., J. Environ. Monit. 7: 475-480, 2005; U.S. Environmental Protection Agency, EPA 815-R-05-002, 2005; Sercu B. et al., Environ. Sci. Technol. 43:293-298, 2009).

For example, the detection of the waterborne protozoans Cryptosporidium or Giardia by method 1623 currently approved by the United States Environmental Protection Agency (USEPA) is characterized by a highly variable and low efficiency of recovery (typically ranging from 10 to 70%) from the filtration membranes and cartridges used for filtration of putatively contaminated samples (DiGiorgio C. L. et al., Appl. Environ. Microbiol. 68: 5952-5955, 2002; Ferguson C. et al., Can J. Microbiol. 50: 675-682, 2004; McCuin R. M. and J. L. Clancy, J. Microbiol. Meth. 63: 73-88, 2005; U.S. Environmental Protection Agency, EPA 815-R-05-002, 2005).

In the case of methods developed for the detection of Cryptosporidium oocysts and Giardia cysts for example, these drawbacks have led investigators to seek for means of improving the performance of this method (Brush C. F. et al., Appl. Environ. Microbiol. 64: 4439-4445, 1998; Hsu B.-M. et al., Water Res. 35: 3777-3783, 2001; McCuin R. M. and J. L. Clancy, Appl. Environ. Microbiol. 69: 267-274, 2003). The lack of efficiency and robustness and the time-to-result-delay (up to fifteen [15] days are required) of Method 1623 may expose human populations to an increase in the risk of large disease outbreaks. Faster and less cumbersome methods for the detection of all waterborne microorganisms, including those that are unculturable or difficult to grow, are therefore warranted. Providing such methods represents an object of the present invention.

Previously, Aldom and Chagla have published a procedure wherein a cellulose acetate membrane is dissolved in organic solvents in order to recover Cryptosporidium oocysts. In this method, the filter is first dissolved in acetone then sequentially exposed to 95% ethanol and 70% ethanol, before resuspension of the residual pellet in eluting fluid, which contains 0.1% Tween 80, 0.1% sodium dodecyl sulfate (SDS), and 0.001% antifoam agent (Sigma Chemical Company, St. Louis, Mo. USA), before the detection of oocysts with the Merifluor Cryptosporidium/Giardia direct immunofluorescence assay kit (Aldom J. E. and A. H. Chagla, Lett. Appl. Microbiol. 20: 186-187, 1995). This method, requiring 4 centrifugation steps of 15 minutes at 650×g, takes 80 minutes to accomplish from filtration to resuspension in eluting fluid. These authors have only reported detection of Cryptosporidium oocysts and the mean recovery of spiked oocysts was 70.5%, calculated from a range of 61-87%.

In 2000, McCuin et al. have evaluated the method of Aldom and Chagla for the recovery of Cryptosporidium parvum and Giardia intestinalis (McCuin R. M. et al., Can. J. Microbiol. 46: 700-707, 2000). On the contrary of the Aldom and Chagla protocol, centrifugation steps of 5 minutes and centrifugal forces of 650, 1050 and 2000×g were used. Moreover, the final pellet was washed with filtered water and compared with eluting fluid. They found that the centrifugation speed did not influence the recovery of G. intestinalis but influenced the recovery of C. parvum where the percentage of recovery was maximal at 2000×g. They also found that the percentage of recovery was higher for both C. parvum and G. intestinalis when filtered water was used to wash the final pellet.

In 2001, Carreno et al. observed a decrease of infectivity for C. parvum oocysts by using a scale-down of the membrane filter dissolution method of Aldom and Chagla (Carreno R. A. et al., Appl. Environ. Microbiol. 67: 3309-3313, 2001). Finally, the Aldom and Chagla dissolution method was used to evaluate the occurrence of Cryptosporidium sp. oocysts in raw sewage and creek water in São Paulo (Farias E. W. C. et al., Braz. J. Microbiol. 33: 41-43, 2002) and to evaluate the occurrence of Giardia cysts in wastewater treatment plants in Italy (Caccio S. M. et al. Appl. Environ. Microbiol. 69: 3393-3398, 2003).

In 1997, Graczyk et al. published a modified version of the original cellulose acetate membrane dissolution in order to improve the recovery rate of Cryptosporidium oocysts (Graczyk T. K. et al., Parasitol. Res. 83: 121-125, 1997). In this method, the sampling bottle was washed with eluting fluid prior to filling it with the sample, in order to prevent the adhesion of a particular matter to the surface. Then, after filtration, an amount of eluting fluid was added to the bottle and filtered like the water sample in order to prevent oocysts carryover between samples. The centrifugal force used was 7000×g to improve the recovery of intact oocysts and the final pellet was not washed with eluting fluid but with filtered water because the detergents could cause loss of oocysts. The mean recovery of spiked oocysts was 78.8%, calculated from a range of 72-82%, whereas the Aldom and Chagla method resulted in a mean recovery of 44.1%, calculated from a range of 24-64%. Graczyk et al. demonstrated that this membrane dissolution method allowed Cryptosporidium oocysts to retain their infectivity (Graczyk T. K. et al., J. Parasitol. 83: 111-114, 1997). The method of Graczyk et al. also allowed the detection of Cyclospora cayetanensis oocysts (Graczyk T. K. et al., Am. J. Trop. Hyg. 59: 928-932, 1998) and Giardia spp. (Graczyk T. K. et al., Parasitol. Res. 85: 30-34, 1999). Finally, the Graczyk et al. dissolution method was used to evaluate the occurrence of Cryptosporidium sp. oocysts in fecal and water samples in Austria (Hassl A. et al. Acta. Trop. 80: 145-149, 2001).

In 1998, the method of Aldom and Chagla was adapted for PCR by Chung et al. (Chung E. et al., J. Microbiol. Meth. 33: 171-180, 1998), Kostrzynska et al. (Kostrzynska M. et al., J. Microbiol. Meth. 35: 65-71, 1999), and Udeh et al. (Udeh P. at al. Mol. Cell. Probes. 14: 121-126, 2000). After resuspension of the residual pellet in the aqueous fluid, 15 to 100% of the volumes were used for DNA extraction and detection by nested PCR (Chung et al. 1998, and Kostrzynska et al. 1999) and QPCR (Udeh et al. 2000). These methods also required centrifugation steps at 650×g, ranging from 200 to 330 minutes from filtration to the beginning of the molecular analysis. Only Cryptosporidium oocysts have been reported to be detectable by these methods. The method of Chung et al. (1998) was used to detect the presence of Cryptosporidium parvum in municipal water samples (Chung E. at al. J. Microbiol. Meth. 38: 119-130, 1999).

The Aldom and Chagla method was also published in a modified version where only methanol was used for dissolving the filtration membrane (Emelko M. B. et al., Proceedings of the 2001 AWWA WQTC, 2001). In order to achieve complete dissolution of the filtration membrane, this method required the use of a sonication step and was only demonstrated for the detection of Cryptosporidium oocysts.

The acetone and methanol (methyl alcohol) that may be used to dissolve cellulose membrane are two solvents also employed for cellular fixation, a step of several histological and histochemical methods used to preserve cells and tissue constituents, by arresting autolysis and decomposition mechanisms. For example, methanol and/or acetone were demonstrated to efficiently fix bacterial cells, protozoan cells or (oo)cysts, insect cells or human cells prior to staining, (immuno)cytochemical or (immuno)histochemical procedures, and isolation of nucleic acids or proteins (Mangels J. I. et al., Diagn. Microbiol. Infect. Dis. 2: 129-137, 1984; Casemore D. P. et al., J. Clin. Pathol. 38: 1337-1341, 1985; Thiriat L. et al., Lett. Appl. Microbiol. 26: 237-242, 1998; Fukatsu T., Mol. Ecol. 8: 1935-1945, 1999; Al-Adhami B. H. et al., Parasitology 133: 555-563, 2006). Methanol is a solvent known to disturb the structure of proteins and to efficiently extract phospholipids and lipopolysaccharides from membranes (Nurminen M. and Vaara M., Biochem. Biophys. Res. Commun. 219: 441-444, 1996; DiDonato D. and Brasaemie D. L., J. Histochem. Cytochem. 51: 773-780, 2003). The action of acetone on cells is similar to that of methanol (Adams C. W. M. and Bayliss O. B., J. Histochem. Cytochem. 16: 115-118, 1968; Paul T. R. and Beveridge T. J., Infect. Immun. 62: 1542-1550, 1994; Hill S. A. and Judd R. C., Infect. Immun. 57: 3612-3618, 1989). Furthermore, it was demonstrated that acetone and methanol can inactivate viruses and various types of cells at certain concentrations, not through complete lysis but most probably by membrane permeabilization (Fauvel M. and Ozanne G., J. Clin. Microbiol. 27: 1810-1813, 1989; Manzenara M. et al., Appl. Environ. Microbiol. 70: 3143-3145, 2004). The notion that acetone and methanol do not lyse cells becomes more understandable when one examines the use of either solvent as membrane permeabilization agent in DNA isolation methods, prior to the lysis achieved by lysozyme or mutanolysin for example (Heath L. S. et al., Appl. Environ. Microbiol. 51: 1138-1140, 1986; Kim Y. M. and Lidstrom M. E., FEMS Microbiol. Lett. 60: 125-130, 1989; Imai T. et al., Anal. Biochem. 222: 479-482, 1994).

The present invention seeks to provide an efficient method for the recovery and concentration of cells and microorganisms.

The present description refers to a number of documents, the content of which is herein incorporated by reference in their entirety.

### SUMMARY OF THE INVENTION

The present invention relates in a first aspect to a method for the recovery of cells or microorganisms from a filtration matrix which comprises or is suspected of comprising the cells or microorganisms.

A purpose of the present invention is to provide a general method for the facilitated and accelerated recovery of membrane-filterable microbial particles including but not limited to metabolically-active or -inactive cells or environmentally-resistant forms ([oo]cysts, spores, etc.) of microorganisms and viruses. An improved filtration membrane dissolution method followed by an efficient confinement (trapping) protocol offers a practical means of recovery of microbial particles in a state compatible with detection procedures.

In accordance with the present invention, the method may comprise the steps of:
a. Obtaining a partially disintegrated filtration matrix from the filtration matrix, and;
b. Treating the partially disintegrated filtration matrix under conditions allowing for its complete dissolution.

The method may also comprise the step of confining the cells or microorganisms in or at the surface of a mobile or static structure or matrix presenting or forming cavities or spaces sufficient to accommodate microbial particles (e.g. for bacteria, 0.5 μm and over). The confinement structure or matrix can also have electrostatic properties.

The present invention therefore relates to an improved method for the dissolution of a filtration membrane (support) for recovery of a microorganism using two organic solvents sequentially and/or detection of such recovered microorganisms. “Detection” is meant to include, for example, cellular and/or molecular detection, namely, detection at the cellular and/or molecular level. Exemplary embodiments of cellular detection may comprise any biochemical, microscopical and/or immunological (for example, without limitation, ELISA, FACS, etc) detection methods know to a person skilled in the art of detecting microorganisms at the cellular level. An exemplary embodiment of molecular detection may include, for example, detection of nucleic acids (DNA, RNA) from recovered microorganisms.

This invention relates to a method for the recovery of microbial particles found in gaseous and liquid matrices tested for the presence of microorganisms.

It is an exemplary embodiment of this invention to use an efficient method for the primary concentration of microbial particles. Capture of microbial particles may be accomplished by filtration on a filtration membrane. In a preferred embodiment said filtration is performed using a cellulose matrix. A further preferred filtration matrix is a cellulose ester membrane.

Primary concentration of the microbial particles may be achieved under conditions allowing partial dissolution or disintegration of the filtration matrix followed by sedimentation (e.g., by centrifugation) of the partially dissolved or disintegrated matrix and then under conditions allowing complete dissolution of the filtration matrix. It is therefore an exemplary embodiment of this invention to provide an efficient method for the recuperation of microbial particles from the concentration receptacle or matrix (for example, a filtration support). In a preferred embodiment said recuperation method involves the dissolution of the filtration matrix with a first organic solvent allowing partial or slow dissolution or disintegration of the filtration matrix. In accordance with the present invention, the partially dissolved or disintegrated matrix carrying the microorganisms may be recovered by sedimentation. The method may further comprise replacing the first organic solvent with a second organic solvent allowing complete dissolution of the filtration matrix. A desirable property of the second organic solvent is that it may be efficient to completely dissolve the filtration matrix using small volumes and provide a solution visually clear of filtration membrane debris. In a further exemplary embodiment, membrane dissolution is performed using methanol followed by acetone.

It is an exemplary embodiment of this invention to provide a secondary confinement for the recuperation of said concentrated microbial particles. In an exemplary embodiment, secondary confinement may be performed using a carrier or a confinement structure or matrix within the confinement receptacle. In another exemplary embodiment, said secondary confinement may be performed on glass beads included in the confinement receptacle. In another preferred embodiment, said glass beads may have dimensions ranging from 150 to 1180 μm. In another embodiment, said glass beads may be used to lyse microbial particles and extract their nucleic acids. The person skilled in the art would know that said secondary confinement may be performed with different confinement structures provided by matrices such as acid-washed glass beads, ceramic spheres, zircon particles, silica spheres, stainless steel beads, tungsten beads, etc. Such matrices are made commercially available by MP Biomedicals (www.mpbio.com) or Adiagene (ww.adiagene.com), for example. Although not necessary, if desired, the confinement matrix may be modified with reagents allowing specific detection of the cells or microorganisms or of molecular components of the cells or microorganism (e.g., antibodies, probes, etc.).

It is an exemplary embodiment of this invention to provide a method for the recovery of microbial particles compatible with molecular amplification and detection methods. In a preferred embodiment of the procedure, said methods may be based on the amplification of nucleic acids. The GenomiPhi™ kit is used for whole genome amplification (WGA) of extracted nucleic acids, but a person skilled in the art would know that other methods, commercially available or not, such as phi29 WGA REPLI-g, whole methylome amplification (WMA), whole transcriptome amplification (WTA), single primer isothermal amplification (SPIA) WGA, molecular displacement amplification (MDA) technology, multiply primed rolling circle amplification (MPRCA), primase-based whole genome amplification (pWGA), and helicase-dependent amplification (HDA) may also be used. In an exemplary embodiment, said methods may be based on the detection of nucleic acids. A person skilled in the art would know that nucleic acid detection methods include without limitation methods such as real-time polymerase chain reaction (rtPCR), quantitative polymerase chain reaction (qPCR), ligase chain reaction (LCR), transcription-mediated amplification (TMA), self-sustained sequence replication (3SR), nucleic acid sequence-based amplification (NASBA), strand displacement amplification (SDA), recombinase polymerase amplification (RPA), loop-mediated isothermal amplification (LAMP), helicase-dependent amplification (HDA), helicase-dependent isothermal DNA amplification (tHDA), branched DNA (bDNA), cycling probe technology (CPT), solid phase amplification (SPA), rolling circle amplification technology (RCA), real-time RCA, solid phase RCA, RCA coupled with molecular padlock probe (MPP/RCA), aptamer based RCA (aptamer-RCA), anchored SDA, primer extension preamplification (PEP), degenerate oligonucleotide primed PCR (DOP-PCR), sequence-independent single primer amplification (SISPA), linker-adaptor PCR, nuclease dependent signal amplification (NDSA), ramification amplification (RAM), multiple displacement amplification (MDA), and/or real-time RAM (Westin L. et al., Nat. Biotechnol. 18:199-204, 2000; Notomi T. et al., Nucl. Acids Res. 28:e63, 2000; Vincent M. et al., EMBO reports 5:795-800, 2004; Piepenburg O. et al., PLoS Biology 4:E204, 2006; Yi J. et al., Nucl. Acids Res. 34:e81, 2006; Zhang D. et al., Clinica Chimica Acta 363:61-70, 2006; McCarthy E. L. et al., Biosens. Biotechnol. 22:126-1244, 2007; Zhou L. et al., Anal. Chem. 79:7492-7500, 2007; Coskun S. and Alsmadi O., Prenatal Diagnosis 27:297-302, 2007; Biagini P. et al., J. Gen. Virol. 88:2629-2701, 2007; Gill P. et al., Diagn. Microbiol. Infect. Dis. 59:243-249, 2007; Lasken R. S. and Egholm M., Trends Biotech 21:531-535, 2003). The scope of this invention is not limited to the use of amplification by PCR technologies, but rather is meant to include the use of any nucleic acid amplification method and/or any other procedure which may be used to increase the sensitivity and/or the rapidity of nucleic acid-based diagnostic tests. The scope of the present invention also covers the use of any nucleic acids amplification and detection technology including real-time and/or post-amplification detection technologies, any amplification technology combined with detection, any hybridization nucleic acid chips or array technologies, any amplification chips or combination of amplification and/or microarray hybridization technologies. Amplification and/or detection using a microfluidic system or a micro total analysis system (μTAS) is under the scope of this invention. In a further preferred embodiment said target amplification method uses whole genome amplification (WGA) followed by PCR or rtPCR.

In an exemplary embodiment, the dissolution of a filtration membrane, generally used for the microbiological analysis of water and other aqueous suspensions, may be followed by secondary confinement using glass beads. Secondary confinement increases the yield of recovery by limiting the loss of microbial particles, including but not limited to vegetative cells or environmentally-resistant forms of indicator, index, or pathogen microorganisms.

This invention relates to a method for the dissolution of a filtration membrane followed by a secondary confinement for the recovery and detection of microbial particles. The method may comprise the steps of:
exposing to methanol (methyl alcohol) a filtration membrane that served to recover microbial particles from a sample, thereby altering the structural integrity of the filter material;
concentrating, isolating, and/or purifying the microbial particles, to remove a significant portion of methanol and methanol-soluble compounds;
further altering the structural integrity of the filter material by exposition to a (small) volume of acetone (dimethyl ketone, 2-propanone);
concentrating, isolating, and/or purifying the microbial particles in the presence of glass beads, to remove a significant portion of acetone and acetone-soluble compounds while improving recovery of microbial particles;
optionally washing the concentrated, isolated, or purified microbial particles, by resuspension in an aqueous solvent to remove the organic solvent(s) and traces of it (them),
optionally concentrating the washed microbial particles, and/or
optionally resuspending and washing the microbial particles in an aqueous solvent, compatible with subsequent detection methods.

In an exemplary embodiment, said filtration membrane may be made of cellulosic material.

In brief, a sample to be analyzed for microbial particles may be filtered through a cellulose filter, on a filtration manifold system or other device well known to those skilled in the art, thereby immobilizing microbial particles at the surface, or trapping them within the pores of the filter. The method of the present invention may be adapted to any filter size or desired porosity. For water analysis, the diameter of filter membranes generally varies from 25 to 293 mm. In a preferred embodiment, a filtration membrane of 47 mm in diameter is used. The porosity of filtration membranes useful for water analysis typically ranges from 0.1 to 0.8 μm. In an exemplary embodiment, a membrane with a porosity of 0.45 μm may be used. The volume of the tested water sample may be dependent on several parameters including the capacity of the filtration membranes, the targeted microorganisms, the type of water, etc. The sample volume typically varies from 10 mL to several litres. The method may be adapted to suit the sample volumes. In an exemplary embodiment, a sample volume of 100 mL, 1L or even higher volume may be tested.

After filtration of an aqueous sample on a system such as a vacuum-operated manifold, the filter may be aseptically removed and transferred to a centrifuge tube or bottle capable of containing the filtration membrane. Depending on the diameter of the filtration membrane, the membrane may be exposed to a volume of methanol and subjected to rapid vigorous mixing, resulting in partial disintegration of the filtration membrane. The volume of methanol may range from 1.0 mL to 50 mL; in an embodiment applicable to membrane filters of 47 mm, 6 to 10 mL of methanol may be used for the disintegration of the membrane in a 15 mL tube. In an exemplary embodiment, the volume of methanol used may be 8.5 mL and rapid mixing by mechanical vortexing may be performed until the membrane visually appears disintegrated into a suspension of flaky bits measuring few millimetres. In an embodiment of the method, desirable filter disintegration is generally obtained within 5 minutes. In an exemplary embodiment, disintegration of a 47 mm cellulose filtration membrane may be observed within 10 seconds.

This disintegration mixture may be centrifuged such that microbial particles and/or membrane debris may be adequately pelleted and the supernatant may be discarded. For microbial particles such as bacteria and parasites, the centripetal force may typically ranges between 1500 and 4000×g for a minimum of 2 minutes. In an exemplary embodiment, a centripetal force of 2100×g is applied for 6 minutes at room temperature (23° C.). Then, the pellet may be exposed to acetone, thereby completing the dissolution of the filter and/or liberating the immobilized or trapped particles and/or microorganisms. The volume of acetone may range from 0.2 mL to 1 mL; in an embodiment applicable to membrane filters of 47 mm in diameter, 0.5 to 1 mL of acetone may be used to dissolve the remnants of membrane after exposition to methanol in a 15 mL tube. In an exemplary embodiment, the microbial particles and membrane remnants may be exposed to 1.0 mL of acetone until membrane remnants visually disappear from the solution. Alternatively, spectroscopic methods and apparatus may be used to determine the clarity (i.e., dissolution) of the filtration membrane.

The microbial particles may be subsequently transferred in tubes containing secondary confinement matrix such as glass beads and pelleted by centrifugation at 15800×g for 3 minutes at room temperature (23° C.) and may be resuspended into an aqueous solution, rendering them amenable to various microbial detection and/or enumeration methods. The nature and volume of aqueous solution may be dependent on the method of detection and/or enumeration. Aqueous solutions may be, for example, water, TE buffer, and phosphate-buffered saline, supplemented or not with detergents.

In the fields of environmental microbiology, water production industry, occupational health, and agri-food, this method may serve to detect microorganisms in air, liquid (e.g., water) or in solid mixed in a liquid or gaseous matrix, for example, by using culture-independent methods such as microscopy, or molecular-based methods relying or not on nucleic acid amplification procedures. Indeed, by relieving physical, electrostatic, and/or hydrophobic interactions between the sought biological particles and/or microorganisms and the polymeric network of a filtration support (e.g. membrane filter), this invention enables or facilitates the rapid, sensitive, and more efficient detection of vegetative cells or environmentally-resistant forms of culturable, unculturable or fastidious index, commensal, or pathogen microorganisms used to assess the microbial safety of, for example, water, food and/or air.

Further scope, applicability and advantages of the present invention will become apparent from the non-restrictive detailed description given hereinafter. It should be understood, however, that this detailed description, while indicating exemplary embodiments of the invention, is given by way of example only.

### DETAILED DESCRIPTION

The present invention therefore relates in a first aspect to a method for the recovery and/or concentration of cells or microorganisms from a filtration matrix which comprises or is suspected of comprising the cells or microorganisms. These cells or microorganisms thus recovered and/or concentrated may be efficiently detected.

In accordance with an embodiment of the invention, the method may comprise the steps of:
a. Obtaining a partially disintegrated filtration matrix from the filtration matrix, and;
b. Treating the partially disintegrated filtration matrix under conditions allowing for its complete dissolution.

In accordance with the present invention the partially disintegrated filtration matrix may be obtained, for example, by partial dissolution of the filtration matrix in a first organic solvent, such as methanol.

In accordance with the present invention, complete dissolution of the partially disintegrated filtration matrix may be obtained by replacing the first organic solvent with a second organic solvent such as acetone. A clear solution comprising the completely dissolved filtration matrix is thus obtained. As indicated herein the volume of this solution may advantageously be minimized in order to concentrate the cells or microorganisms.

Replacement of the first organic solvent may be performed by sedimenting the partially disintegrated filtration matrix and removing the first organic solvent while leaving the pellet as intact as possible. A desired volume of the second organic solvent may then be added.

In an exemplary embodiment, the filtration matrix may be a cellulose ester filter such as a mixed cellulose ester filter.

The method of the present invention may further comprise a step of confining the cells or microorganisms in a mobile or static confinement structure or matrix. Such confinement may be performed, for example, by centrifugating the clear solution comprising the completely dissolved filtration matrix over beads.

The second organic solvent may then be replaced with a buffer suitable for a detection method.

In accordance with another embodiment of the invention, the method may comprise the steps of:
a. Obtaining a partially disintegrated filtration matrix from the filtration matrix by treating the filtration matrix under conditions allowing for its partial disintegration;
b. Treating the partially disintegrated filtration matrix under conditions allowing for its complete dissolution thereby obtaining a completely dissolved filtration matrix, and;
c. Confining the cells or microorganisms in a mobile or static confinement structure or matrix by contacting the completely dissolved filtration matrix with the confinement structure or matrix.

The partially disintegrated filtration matrix may be obtained by treating the filtration matrix with a suitable solvent (and/or contacting the filtration matrix with the solvent for a suitable period of time), sedimenting the resulting partially disintegrated filtration matrix and removing the solvent.

The completely dissolved filtration matrix may be obtained by treating the partially disintegrated filtration matrix with a suitable solvent (and/or contacting the partially disintegrated filtration matrix with the solvent for a suitable period of time) until the solution becomes essentially clear (i.e., as determined by spectrophotometric methods, by visual observation or else). The volume of solvent used at this step may be minimized (e.g., less than 1.5 ml) to obtain a concentrated preparation of cells or microorganisms.

The cells or microorganisms may then be captured and/or further concentrated, for example, by centrifugating the solution of completely dissolved filtration matrix over a confinement structure or matrix such as beads. The cells or microorganisms then are confined in spaces between beads and the supernatant may be removed thereby leaving a highly concentrated preparation of cells or microorganisms.

This highly concentrated preparation of cells or microorganisms may be used for any desired purposes, such as for detection purposes by light or electron microscopy, microbiological or cellular cultivation, cytometry or fluorocytometry, antigen detection using antibody(ies) or aptamer(s), or molecular detection of protein or nucleic acid biomarkers. For example, the resulting highly concentrated preparation of cells or microorganisms may be lyzed (e.g., with the help of the beads) to liberate the nucleic acids which are then detected using methods that are known to those of skill in the art.

The method of the present invention may be applied to recover or concentrate bacterial or archaeal (procaryotic), or eucaryal (eucaryotic) cells from a filtration matrix.

In an exemplary embodiment, the microorganisms may be selected from the group consisting of bacterium, bacterial spores, protozoans, protozoan cysts or oocysts, multicellular parasites, fungi, fungal spores, viruses, and bacteriophages.

The method of the present invention may more particularly be used to recover a bacterium such as Bacillus atrophaeus subsp. globigii or a spore thereof from an original (i.e., initial) sample comprising the bacterium.

In another example, the method of the present invention may also particularly be used to recover a bacterium or a spore thereof from an original sample. Bacterium that may advantageously and efficiently be recovered may comprise, without limitation, those which are selected from the group consisting of E. coli, Enterococcus sp, E. faecium and E. faecalis or a spore thereof.

Alternatively, the method of the present invention may also be used to recover protozoan or protozoan (oo)cyst including for example and without limitation Cryptosporidium parvum, C. hominis (also known as C. parvum human genotype) Giardia intestinalis (synonym of G. lamblia and G. duodenalis) or (oo)cysts thereof.

In accordance with the present invention, the microorganisms may be recovered from a gaseous or liquid matrix. For example, the method may allow recovery and concentration of microorganisms from potable water, natural water reservoirs, sewage, well water or water from treatment plant.

It has been found that the method of the present invention allows for the recovery of almost each microorganism and each microorganism species found in the original sample.

For example, the method allows for the recovery of at least 1 cfu or 1 microorganism, at least 2 cfu or 2 microorganisms, at least 5 cfu or 5 microorganisms, at least 10 cfu or 10 microorganisms, at least 20 cfu or 20 microorganisms, at least 50 cfu or 50 microorganisms, of at least 100 cfu or 100 microorganisms or more per tested volume.

The method of the present invention also allows for the recovery of multiple microorganisms species from a single sample.

Detection of multiple microorganism species may also simultaneously be performed therefore reducing the time required to determine whether or not the sample contains a specific microorganism.

The present invention also provides in another aspect, a kit for the recovery of cells or microorganisms from a filtration matrix which comprises or is suspected of comprising cells or microorganisms. The kit may comprise: a first vial comprising a first organic solvent capable of partially disintegrating the filtration matrix such as, for example, methanol and a second vial comprising a second organic solvent capable of completely dissolving the filtration matrix such as, for example, acetone.

In accordance with the present invention the kit may further include a vial comprising a mobile or static confinement structure or matrix (e.g., beads).

Also in accordance with the present invention, the kit may further comprise a filtration matrix (e.g., a cellulose ester filter).

Further in accordance with the present invention, the kit may further comprise reagents for the specific detection of cells or microorganisms by light or electron microscopy, microbiological or cellular cultivation, cytometry or fluorocytometry, antigen detection using antibody(ies) or aptamer(s), and molecular detection of protein or nucleic acid biomarkers using nucleic acid amplification methods, in situ molecular hybridization, or molecular hybridization onto protein or nucleic acid biochips/microarrays.

The kit may also comprise instructions for the primary confinement, e.g., the sequential partial disintegration of the filtration matrix and complete dissolution of the filtration matrix as well as instructions for the secondary confinement (e.g., confinement using a mobile or static structure or matrix).

In the present invention, cellulose membrane filtration will be referred to as any filtration methods using flat, stacked, pleated, or tangential membrane supports, utilized either solely in a reusable or disposable membrane support and/or encased in specialized cartridges. Matrices analyzed by filtration methods may be any aqueous and gaseous matrices including but not limited to any finished, fresh, marine, or estuarine water used for drinking, food processing, recreation, propagation of fish, shellfish, or wildlife, agriculture, industry, building support, navigation, or as source water for drinking (U.S. Environmental Protection Agency, Fed. Register 68: 43272-43283, 2003), food extracts, air samples, medical gases, etc.

Filtration membranes and membrane filtration system. The membrane dissolution protocol was developed using hydrophilic mixed cellulose ester membrane filters. Several commercially-available membrane filters, with different diameters and nominal porosities were tested. Membranes with diameters of 25 and 47 mm and with porosities of 0.2, 0.45, and 0.8 μm were tested. In an exemplary embodiment adapted for the filtration of bacteria and parasites, plain and gridded sterile Metricel GN-6 (Pall Canada, Mississauga, ON, Canada), as well as plain and gridded sterile SO-Pak (also known as EZ-Pak) filter membranes (Millipore Canada, Mississauga, ON, Canada) having a diameter of 47 mm and a pore size of 0.45 μm were tested. The diameter and porosity of the membrane may be adjusted to the size of the filtration system, device and/or to the pore size needed to optimally immobilize and/or entrap sought microbes and/or microbial particles.

The determination of the microbiological quality of water is generally assessed with volume samples ranging from 10 mL to more than 1000 L, depending on the quality of the water, ranging from sewage to finished water entering a distribution system. Several filtration apparatuses are commercially available and known to those skilled in the art. For the determination of the microbiological quality of water, 3-place stainless steel membrane filtration manifold equipped with 650-mL stainless steel funnels (Millipore Canada, Mississauga, ON, Canada) was used to test water samples volumes ranging from 10 to 1000 mL. In an exemplary embodiment of the invention, representative of current procedures for the determination of bacteria and/or fecal contamination indicators in water, the sample volume tested was 100 mL. In another exemplary embodiment of the invention, representative of procedures for the determination of fecal contamination indicators, index microorganisms, or pathogens in water, the sample volume tested was between 100 and 1000 mL. The stainless steel funnels were sterilized with a ultraviolet light (UV) sterilizer (Millipore Canada, Mississauga, ON, Canada) and vacuum-aided filtration was accomplished using a chemical duty vacuum pump (Millipore Canada, Mississauga, ON, Canada). These devices were used in accordance with the manufacturer's instructions. As membrane filtration is applicable to the filtration of smaller or larger volumes of aqueous or gaseous samples compatible with mixed cellulose ester membranes and considering that many types of membrane filtration devices have been developed for these applications, it is understood that the nature and capacity of the filtration system or device may be adjusted accordingly. The procedure may be adaptable to both microscale and macroscale usage.

Primary Confinement

Partial disintegration of the filtration membrane. Mixed cellulose ester filtration membranes of a diameter of 47 mm and a pore size of 0.45 μm were used. As the sample volume and the nature of contaminants may vary, it is understood that the diameter and pore size of the filtration membrane, the volume of reagents, and the length of incubation periods may vary. Thus, following the filtration of an aqueous sample chemically compatible with mixed cellulose ester membranes, the filtration membrane was aseptically removed from the filtration system or device with flame-sterilized forceps and transferred to a sterile container appropriate for the size of the membrane. For membranes of 47 mm in diameter, reaction tubes of 10 to 50 mL may be used.

In an exemplary embodiment of the invention, the membrane was transferred to a 15 mL polypropylene tube (Sarstedt, Newton, N.C., U.S.A.). The filtration membrane was exposed to 6 to 10 mL of methanol (methyl alcohol) for a period of time ranging from 1 to 300 seconds. In a preferred embodiment of the invention, a 47 mm membrane is exposed for 10 seconds to 8.5 mL of methanol and the disintegration of the filtration membrane is accelerated by vigorous agitation for 10 seconds on a vortex mixer set at maximum speed. After this step, the reaction tube and its content were centrifuged for a minimum of 2 minutes at 1500 to 4100×g. In an exemplary embodiment of the invention, this step is performed for 6 minutes at 2100×g in a benchtop centrifuge maintained at room temperature (23° C.). The supernatant is removed using a micropipettor and discarded, with care taken not to disturb the pellet.

Complete dissolution of the filtration membrane. The action of methanol on mixed cellulose ester membranes leaves certain residues. To complete the dissolution of the filtration membrane, a small amount of histological-grade acetone (dimethyl ketone or 2-propanone; EMD Chemicals, San Diego, Calif., U.S.A.) was added to the pellet and the treatment was accelerated by vigorous agitation (10 seconds at maximum setting) on a vortex mixer. For a 47 mm 0.45 μm membrane, the volume of acetone used in a 15 mL polypropylene tube ranged from 0.5 to 9.0 mL. In a preferred embodiment of the invention, the microbial particles and membrane remnants were exposed to 1.0 mL of acetone until dissolution was completed, as determined by visual observation of the disappearance of filter material.

Secondary Confinement.

After complete dissolution, the resulting clear acetone suspension was transferred to a 2.0 mL microcentrifuge tube containing acid-washed glass beads (150-212 μm and 710-1180 μm; Sigma-Aldrich, St. Louis, Mo., U.S.A.) and centrifuged for 3 minutes at 15800×g in a microcentrifuge maintained at room temperature (23° C.). The supernatant was removed using a micropipettor and discarded, with care taken to minimize glass beads agitation, leaving approximately 20 μL of supernatant. This supernatant and pellet may further be processed for histological or immunological analysis or to extract microbial nucleic acids, for example. The 15 mL polypropylene tube used during the secondary dissolution step may be briefly rinsed with a small volume of acetone. This rinsing volume may be added to the vessel or tube that served to collect and/or concentrate the first acetone-based dissolution mixture potentially containing cells released by the membrane dissolution procedure. If desired, the glass beads used in the confinement procedures may further be used for the cell lysis procedure. In an exemplary embodiment of the invention, 1.0 mL of histological-grade acetone was used to rinse the 15 mL polypropylene tube and the resulting rinsing solution was carefully added to the pelleted dissolution mixture contained in the 2.0 mL microcentrifuge tube with glass beads.

The resulting solution was centrifuged for 3 minutes at 15800×g in a microcentrifuge maintained at room temperature (23° C.) and the resulting pellet was washed with an aqueous solvent suitable for the non culture-based detection of the sought cells. In an exemplary embodiment of the invention, the pellet was gently washed with 1.0 mL of TE (Tris-HCl 100 mM, EDTA 1 mM, pH 8.0), taking care to minimize glass bead agitation, and centrifuged for 3 minutes at 15800×g in a microcentrifuge maintained at room temperature (23° C.). After centrifugation, the supernatant was removed using a micropipettor and discarded with care taken not to disturb the pellet, leaving approximately 10 μL of aqueous supernatant on top of the glass bead pellet. This supernatant and pellet can be further processed for histological and/or immunological analysis and/or to extract microbial nucleic acids.

The method of the present invention allows the recovery of microbial particles by primary confinement based on the partial disintegration followed by complete dissolution of the membrane filter and secondary confinement using glass beads. Since we are capable of washing and recovering microbial particles by low-speed centrifugation following dissolution, the present invention does not directly lead to cellular lysis which may be achieved, if required, by mechanical or ultrasonic actuation, or the addition of chemicals and/or enzymes. This characteristic of the procedure opens the possibility of detecting unculturable, metabolically inactive, and/or damaged (pathogen) cells not easily recovered by culture-based methods but that could revert to a disease-causing state upon ingestion (Gostin L. O. et al., Am. J. Public Health 90: 847-853, 2000; Sylvester D. M. et al., Infect. Dis. Rev. 3: 70-82, 2001; Nwachcuku N. and Gerba C. P., Curr. Opin. Biotechnol. 15: 175-180, 2004).

The method of the present invention may be adapted to remove some unwanted contaminant(s) trapped by membrane filters. For example, washing filter membranes with a solution containing a compound such as PVP 360 has been used to reduce humic acids and other PCR inhibitor substances from environmental water samples (Guy R. A. et al., Appl. Environ. Microbiol. 69: 5178-5185, 2003). Such washing steps performed prior to the membrane dissolution procedure are under the scope of this invention.

Other aspects of the invention relates to methods for the recovery of a microorganism from a filtration support which may comprise, for example, the step of dissolving the filtration support using at least two organic solvents sequentially.

In a further aspect, the present invention provides a method for the recovery of a microorganism from a filtration support which may comprise the steps of
a) dissolving the filtration support, and
b) confining the microorganism in a solid matrix.

The method may also further comprise the step of lysing the microorganism.

In yet a further aspect, the present invention provides a method for the recovery and molecular detection of a microorganism from a filtration support, which may comprise the steps of:
a) dissolving the filtration support,
b) confining the microorganism in a solid matrix,
c) lysing the microorganism, and
d) detecting nucleic acids from the lysed microorganism.

In accordance with a first embodiment of the invention, the filtration support may be dissolved using at least one organic solvent.

In accordance with a second embodiment of the invention, the filtration support may be dissolved using two organic solvents.

The two organic solvents may be used, for example, in a sequential manner.

In accordance with the present invention methanol may be used as a first organic solvent and acetone may sequentially be used as a second organic solvent.

In accordance with an embodiment of the invention, the microorganism may be selected, for example, from the group consisting of bacterium (e.g., gram positive bacterium, gram negative bacterium), bacterial spores (e.g., bacterial endospore, bacterial exospore), archaea, protozoan, parasite, parasite oocyst, parasite cyst, parasite spore, microsporidia, fungi, fungal endospore, fungal exospore, virus, and bacteriophage.

The filtration support may be composed of cellulosic material such as cellulose ester or mixed cellulose ester.

In an embodiment of the invention, the solid matrix may comprise a mobile confinement structure or matrix, such as glass beads.

In an additional aspect, the present invention provides a method for the recovery and cellular detection of a microorganism from a filtration support, where the method may comprise the steps of:
a) dissolving the filtration support using at least two organic solvents sequentially, and
b) detecting the microorganism at the cellular or molecular level.

Cellular detection may be performed by methods known in the art including, for example, biochemical detection, microscopy detection and immunological detection.

In yet an additional aspect, the method of the present invention provides for the recovery and molecular detection of a microorganism from a filtration support, where the method may comprise the steps of:
a) dissolving the filtration support using at least two organic solvents sequentially,
b) lysing the microorganism, and
c) detecting nucleic acids from the lysed microorganism.

### EXAMPLE 1

### Determination of Effective Organic Solvent Combination for Dissolution of Mixed Cellulose Ester Filtration Membranes

The use of methanol and acetone, alone, mixed, or used consecutively, differentially impact on the integrity of cellulose ester filtration membranes was studied herein.

Materials and Methods

For each experiment, one cellulose mixed esters filtration membrane of each type tested (plain and gridded Pall Metricel GN-6 as well as Millipore SO-Pak, diameter of 47 mm and pore size of 0.45 μm) was introduced into a 50 mL tube and exposed for 2 minutes (including vortexing at maximum speed) to 10 mL of either methanol (Method A), acetone (Method B), or methanol-acetone 1:1 (Method C), before the slurry was centrifuged for 3 minutes at 2100×g. The supernatant was removed and the pellet was resuspended into 10 mL of phosphate-buffered saline (PBS; 137 mM NaCl, 6.4 mM Na₂HPO₄, 2.7 mM KCl, 0.88 mM KH₂PO₄, pH 7.4).

In the “acetone followed by methanol” (Method D) and “methanol followed by acetone” (Method E or MFA) tests, the filtration membrane was introduced into a 50 mL tube, exposed for 2 minutes to the first solvent of the combination (including vortexing at maximum speed) before the slurry was centrifuged for 3 minutes at 2100×g. The supernatant was removed, and subsequently, the pellet was exposed for 2 minutes (including vortexing at maximum speed) to 10 mL of the second solvent of the combination. The resulting slurry was collected by centrifugation, the supernatant was removed and the content of each tube was resuspended into 10 mL of PBS.

The “Aldom and Chagla” procedure (Method F) was performed as previously described (Aldom J. E. and A. H. Chagla, Lett. Appl. Microbiol. 20: 186-187, 1995). In brief, the filtration membrane was exposed for 2 minutes to 10 mL of acetone (including vortexing at maximum speed) before the slurry was centrifuged for 3 minutes at 2100×g. The supernatant was removed and the pellet was resuspended and exposed for 2 minutes to 10 mL of ethanol 95%. The resulting slurry was centrifuged for 3 minutes at 2100×g, the supernatant was removed and the pellet was resuspended and exposed for 2 minutes to 10 mL of ethanol 70%. This slurry was collected by centrifugation, the supernatant was removed and the content of the tube was resuspended into 10 mL of eluting fluid (PBS containing 0.1% Tween 80, 0.1% SDS, and 0.001% Antifoam A).

Results

After exposing the solvent on the Metricel GN-6 and SO-Pak membranes and resuspending the centrifuged materials in PBS, the following was observed:

Method A. Dissolution in the presence of methanol only. The aqueous (PBS) solution was turbid and whitish while a white pellet composed of fine particles was present. Addition of methanol did not show a complete dissolution of the membrane.

Method B. Dissolution in the presence of acetone only. The aqueous solution was clear with no visible residue. A minimum of 4 mL of acetone was required to obtain a total dissolution of the membrane.

Method C. Dissolution in the presence of methanol-acetone 1:1. The aqueous solution was clear with floating aggregated translucent membrane residues. Addition of methanol-acetone 1:1 did not show a complete dissolution of the membrane.

Method D. “Acetone followed by methanol” dissolution. The resulting aqueous solution was clear with no visible residue. A minimum of 4 mL of acetone was required to obtain a total dissolution of the membrane.

Method E. “Methanol followed by acetone” (MFA) dissolution. Addition of methanol lead to solution having the same properties as in Method A. After sedimenting the membrane particles and replacing methanol with acetone, the resulting aqueous solution was clear with no visible residue. A minimum of 0.5 mL of acetone was required to obtain a total dissolution of the membrane.

Method F. Aldom and Chagla's method. The resulting aqueous solution was clear with no visible residue. A minimum of 4 mL of acetone was required to obtain a total dissolution of the membrane.

This experiment demonstrates that acetone alone or sequentially with methanol lead to the complete dissolution of cellulose ester filtration membranes. Results from Method E show that the MFA procedure leads to the complete dissolution of the cellulosic material of the filtration membrane. This procedure involves exposing the membrane to methanol, followed by removal of most of this organic solvent before completing the dissolution of the cellulose membrane in the presence of acetone. Approximately 230 μL of acetone is required for dissolving 1 cm² of filter support material. A first exposure to methanol reduces this volume to approximately 29 μL of acetone which corresponds to more than 8 times less acetone. Methods B, D, and F also dissolved the filtration membrane efficiently albeit a larger volume of acetone was required.

The first step of Method E uses methanol which yielded a pellet composed of fine filter particles. These particles which may assist (might be helpful in) the confinement of microbial particles within the pellet. The resulting pellet is dissolved with a minimal volume of acetone. This acetone solution containing microbial particles may be pelleted over a secondary confinement matrix such as glass beads as described in the following examples. Such a confinement structure or matrix may facilitate the recovery of microbial particles in a state where detection procedures are applicable.

### EXAMPLE 2

### Recovery of Escherichia coli, and Enterococcus faecalis, Cryptosporidium parvum oocysts, Giardia intestinalis cysts and Bacillus atrophaeus subsp. globigii Spores by Membrane Dissolution/Glass Beads Confinement Procedure and Detection by WGA-rtPCR

E. coli and enterococci are indicators of the fecal contamination of water. The recovery and/or detection of membrane-filtered water containing E. coli and/or E. faecalis after dissolution of the membrane made of cellulose mixed esters followed by the glass beads confinement procedure is studied in this example.

The recovery of Cryptosporidium and Giardia encysted particles from putatively contaminated water samples is currently a cumbersome, lengthy, and relatively expensive process complicated by electrostatic interactions between microbial particles and the filtration matrix (Drozd C. and J. Schwartzbrod, Appl. Environ. Microbiol. 62: 1227-1232, 1996; Carey C. M. et al., Water Res. 38: 818-862, 2004). The method of Aldom and Chagla (Aldom J. E. and A. H. Chagla, Lett. Appl. Microbiol. 20: 186-187, 1995) contributed to partly alleviate this complication but has not become part of current detection procedures. This method presents a highly variable recovery rate of C. parvum ranging from 0 to 90% (McCuin R M et al., Can. J. Microbiol. 46: 700-707, 2000). The feasibility of a rapid and sensitive detection of waterborne parasites retained onto a filtration membrane made of mixed cellulose esters was studied herein. C. parvum and G. intestinalis, two microorganisms that are released in water as encysted forms (oocysts or cysts) that cannot be easily cultivated were used while B. atrophaeus subsp. globigii spores served as control.

Materials and Methods

Filtration membranes. The membrane dissolution followed by a glass beads confinement procedure was performed with plain sterile Metricel GN-6 mixed cellulose esters membrane filters (47 mm diameter, porosity of 0.45 μm; Pall Canada, Mississauga, ON, Canada).

Bacteria and methods used for culture-based testing of membrane-filtered samples. The bacterial strains studied for this application were E. coli ATCC 11775 and E. faecalis ATCC 19433. For spiking experiments, bacterial strains grown from frozen stocks kept at −80° C. in brain heart infusion (BHI) medium containing 10% glycerol, were cultured on sheep blood agar or in BHI broth. Following membrane filtration, E. coli was tested by the culture-based USEPA Method 1604 (U.S. Environmental Protection Agency, EPA 821-R-02-024, 2002), performed on MI solid medium (BD Diagnostic Systems, Sparks, Md., U.S.A.) supplemented with 5 μg/mL of cefsulodin (Sigma-Aldrich, St. Louis, Mo., U.S.A.). Following membrane filtration on Metricel GN-6 membranes, E. faecalis was tested by the culture-based USEPA Method 1600 (U.S. Environmental Protection Agency, EPA 821-R-02-022, 2002), performed on mEI medium (BD Diagnostic Systems, Sparks, Md., U.S.A.).

Cryptosporidium oocysts and Giardia cysts. Freeze-killed C. parvum Iowa isolate oocysts and G. lamblia (syn. G. intestinalis, G. duodenalis) Human isolate H-3 cysts were obtained from Waterborne Inc. (New Orleans, La., U.S.A.).

Preparation of spiked water samples. Spiked samples were prepared in commercially available spring water (Labrador, Ville d'Anjou, Québec, Canada). Cultures of E. coli or E. faecalis cells grown to logarithmic phase (0.5-0.6 OD_{600 nm}) were adjusted to a 0.5 McFarland standard, before being serially diluted ten-fold in PBS. Aliquots of the 10⁻⁵ dilution of E. coli and E. faecalis were used to prepare the spiked water samples. Particle counts provided by the supplier of Cryptosporidium oocysts and Giardia cysts were confirmed by counting with Petroff-Hauser chambers. Aliquots of both particle types were used to prepare spiked water samples also containing bacterial cells.

Aliquots of bacterial cell dilutions and (oo)cyst preparations were spiked in spring water to produce suspensions targeting 16, 8, 4, 2, and 1 of each microbial particle type per 100 mL. The number of spiked bacteria (cfu per 100 mL) was estimated by plate count procedures, in multiple replicates on sheep blood agar, and MI or mEI media. Molecular detection was achieved by the WGA-rtPCR procedure described below.

An internal process control, typically consisting of approximately 60 B. atropheus subsp. globigii spores was added to each spiked 100 mL water sample prior to filtration. The methods and reagents for preparing B. atropheus subsp. globigii spores and detecting their nucleic acids is fully described elsewhere (International patent application number PCT/CA2003/01925 and Picard F. J. et al., J. Clin. Microbiol. 47: 751-757, 2009).

Membrane filtration of a water sample. Briefly, a plain sterile Metricel GN-6 filtration membrane was aseptically positioned on a filtration head of the manifold and secured in place by installing a UV-sterilized stainless steel funnel. A 3-place stainless steel membrane filtration manifold equipped with 650-mL stainless steel funnels (Millipore Canada, Mississauga, ON, Canada) was used. The sample to be tested was poured in the funnel and vacuum provided by a pump was applied to allow the sample to flow through the filtration membrane, according to the instructions of the manufacturer. After the passage of a spiked water sample of 100 mL, the funnel was rinsed with 20 mL of sterile water and the resulting solution/suspension was also flowed through the filtration membrane.

Partial disintegration of the filtration membrane and primary confinement. Following filtration, the filtration membrane was aseptically removed from the filtration manifold with flame-sterilized forceps and transferred to a sterile 15 mL polypropylene tube (Sarstedt, Newton, N.C., U.S.A.). The filtration membrane was exposed for 10 seconds to 8.5 mL of HPLC-grade methanol (methyl alcohol; Sigma-Aldrich, St. Louis, Mo., U.S.A.) and disintegration was accelerated by vigorous agitation on a vortex mixer (10 seconds at maximum speed). After this step, the reaction tube and its content were centrifuged for 6 minutes at 2100×g in a benchtop centrifuge maintained at room temperature (23° C.). The supernatant was removed using a micropipettor and discarded with care taken not to disturb the pellet. This disintegration step yielded incomplete and/or partial dissolution of the membrane.

Complete dissolution of the filtration membrane and secondary confinement. One (1) mL of histological-grade acetone (EMD Chemicals, San Diego, Calif., U.S.A.) was added to the pellet and complete dissolution was achieved by vigorous agitation on a vortex mixer (10 seconds at maximum speed). After complete dissolution, the resulting clear acetone suspension putatively containing the cells released from the filtration membrane was transferred to a tube (2.0 mL microcentrifuge tube) containing a mixture of acid-washed glass beads. The suspension was centrifuged for 3 minutes at 15800×g in a microcentrifuge maintained at room temperature (23° C.) and the supernatant was removed using a micropipettor and discarded, with care taken to minimize glass bead agitation, leaving approximately 20 μL of solvent supernatant.

Rinsing of recovered microbial particles and removal of organic solvent(s). To maximize the recovery of filtered cells, the 15 mL polypropylene tube was briefly rinsed with 1.0 mL of histological-grade acetone and the resulting mixture was transferred to the tube that served to collect and/or concentrate the first acetone-based membrane dissolution mixture with microbial cells released by the procedure. The resulting solution was centrifuged for 3 minutes at 15800×g in a microcentrifuge maintained at room temperature (23° C.). The resulting pellet was washed with 1.0 mL of TE (Tris-HCl 100 mM, EDTA 1 mM, pH 8.0) and centrifuged for 3 minutes at 15800×g in a microcentrifuge maintained at room temperature.

Lysis procedure for extraction of microbial nucleic acids. After centrifugation of the washed filtrate-glass beads suspension in the presence of TE buffer, the supernatant was removed using a micropipettor and discarded, with care taken to minimize glass bead agitation, leaving a residual volume of approximately 10 μL on top of the glass beads. Forty (40) μL of GenomiPhi™ sample buffer (part of the illustra GenomiPhi™ DNA Amplification Kit; GE Healthcare, Montreal, Québec, Canada) was added to the reaction mixture and the lysis of the cells contained in the pellet was achieved by vigorous mixing, at maximum speed, on a vortex mixer for 5 minutes at room temperature (23° C.). After a quick spin in a microcentrifuge, the reaction tube containing the cell lysate was incubated 3 minutes at 95° C., then briefly spun in a microcentrifuge, and kept on ice for a minimum of 3 minutes.

Whole genome amplification (WGA) procedure. A mixture of forty-five (45) μL of GenomiPhi™ reaction buffer and 4 μL of Phi29 (φ29) DNA polymerase (illustra GenomiPhi™ DNA Amplification Kit) were added to the lysate, gently mixed by finger tapping, before being briefly spun in a microcentrifuge. The WGA reaction mixture was incubated for 3 hours at 30° C. The enzymatic reaction was then inactivated by 10-minute incubation at 65° C.

Real-time PCR (rtPCR) amplification of WGA-amplified nucleic acids. For specific or generic rtPCR amplification of sought microorganisms, the primers and dual-labeled (TaqMan) detection probes used are described in Table 1. One (1) μL of the WGA reaction mixture was transferred directly to a 24 μL rtPCR mixture containing 50 mM KCl, 10 mM Tris-HCl (pH 9.1), 0.1% Triton X-100, 2.5 mM MgCl₂, 200 μM each deoxyribonucleoside triphosphate (dNTP; GE Healthcare, Baie d'Urfé, Québec, Canada), 3.3 μg/μL of bovine serum albumin (BSA; Sigma-Aldrich Canada Ltd., Oakville, Ontario, Canada), 0.025 enzyme unit (U) of Taq DNA polymerase (Promega, Madison, Wis., U.S.A.) combined to TaqStart antibody (Clontech, Palo Alto, Calif., U.S.A.). Independent rtPCR mixtures also contained 0.4 μM of each PCR amplification primer for E. coli (SEQ ID 1-2); for Enterococcus sp. (SEQ ID 4-5); for C. parvum (SEQ ID 13-14); for G. intestinalis (SEQ ID 16-17); for B. atrophaeus subsp. globigii (SEQ ID 19-20); and for m13pSL3 (SEQ ID 22-23), 0.2 μM of each dual-labeled (TaqMan) detection probe for E. coli (SEQ ID 3); for Enterococcus sp. (SEQ ID 6); for C. parvum (SEQ ID 15); for G. intestinalis (SEQ ID 18); and for B. atrophaeus subsp. globigii (SEQ ID 21). The PCR mixtures were subjected to thermal cycling with a Rotor-Gene 3000 (Corbett Life Sciences, now QIAGEN Inc., Mississauga, Ontario, Canada) under the conditions presented in Table 2.

Amplification of internal control (m13pSL3). To examine the inhibitory potential of a concentrated, extracted, and/or amplified sample, one (1) μL of the WGA reaction mixture was transferred directly to a 19 μL PCR mixture containing 50 mM KCl, 10 mM Tris-HCl (pH 9.0), 0.1% Triton X-100, 2.5 mM MgCl₂, 0.4 μM of each PCR amplification primer (SEQ ID 22-23), 200 μM each deoxyribonucleoside triphosphate (dNTP; GE Healthcare, Baie d'Urfé, Québec, Canada), 3.3 μg/μL of bovine serum albumin (BSA; Sigma-Aldrich Canada Ltd., Oakville, Ontario, Canada), 0.025 enzyme unit (U) of Taq DNA polymerase (Promega, Madison, Wis., U.S.A.) combined to TaqStart antibody (Clontech, Palo Alto, Calif., U.S.A.), and 1000 copies of m13pSL3 plasmid. The PCR mixture was subjected to thermal cycling with a PTC-200 DNA Engine thermocycler (MJ Research Inc. Watertown, Mass., U.S.A.) under the following conditions: 3 min at 95° C. and then 40 cycles consisting of a denaturation step of 1 sec at 95° C., an annealing step of 30 sec at 58° C. for m13pSL3 and of an extension step of 30 sec at 72° C. (Lansac N. et al., Eur. J. Clin. Microbiol. Infect. Dis. 19: 443-451, 2000). For estimating the inhibitory potential of a sample, the amplification products of the internal control plasmid was visualized by agarose gel analysis, as previously described (Martineau F. et al., J. Clin. Microbiol. 36: 618-623, 1998).

TABLE 1
PCR primers and dual-labeled (TaqMan) detection probes

| | | | | Amplicon |
|---|---|---|---|---|
| Target | | | | size |
| organism | SEQ | | Nucleotide | (base |
| (target gene) | ID | Name | sequence (5' → 3') | pairs) |
| E. coli (tuf)¹ | 1 | TEcol553 | TGGGAAGCGAAAATCCTG | na* |
| | 2 | TEcol754 | CAGTACAGGTAGACTTCTG | |
| | 3 | TEcoB573-T1-B1 | TET-AACTGGCTGGCTTCCTGG-BHQ-1 | |
| Enterococcus | 4 | ECST784F | AGAAATTCCAAACGAACTTG | na* |
| sp. (16S | 5 | ENC854R | CAGTGCTCTACCTCCATCATT | |
| rRNA)² | 6 | GPL813PQ | FAM-TGGTTCTCTCCGAAA- | |
| | | | TAGCTTTAGGGCTA-BHQ-1 | |
| E. faecalis | 7 | Mefs569 | GAACAGAAGAAGCCAAAAAA | na* |
| (mtlF)-E. | 8 | Mefs670 | GCAATCCCAAATAATACGGT | |
| faecium (ddl)³ | 9 | Defm273 | TGCTTTAGCAACAGCCTATCAG | |
| | 10 | Defm468 | TAACTTCTTCCGGCACTTCG | |
| | 11 | Mefs-TL1-A1 | FAM-CA^{L}GGAAT^{L}CTGT- | |
| | | | ^{L}GTA^{L}GTG^{L}CAAG-BHQ-1** | |
| | 12 | Defm-T1-F2 | CalFluorRed610-CTCGAGCAATC- | |
| | | | GTTGAACAAGGAATTG-BHQ-2 | |
| C. parvum | 13 | COWPP702F | CAAATTGATACCGTTTGTCCTTCTG | na* |
| (COWP)⁴ | 14 | COWPP702R | GGCATGTCGATTCTAATTCAGCT | |
| | 15 | COWPP702P | FAM-TGCCATACATTGTTGT- | |
| | | | CCTGACAAATTGAAT-BHQ-1 | |
| G. intestinalis | 16 | GiardinP241F | CATCCGCGAGGAGGTCAA | na* |
| (Giardin)⁴ | 17 | GiardinP241R | GCAGCCATGGTGTCGATCT | |
| | 18 | GiardinP241P | FAM-AAGTCCGCCGACAA- | |
| | | | CATGTACCTAACGA-BHQ-1 | |
| B. atrophaeus | 19 | ABgl158 | CACTTCATTTAGGCGACGATACT | na* |
| subsp. globigii | 20 | ABgl345a | TTGTCTGTGAATCGGATCTTTCTC | |
| (atpD)⁵ | 21 | ABgl-T1-A1 | FAM-CGTCCCAATGTTACATTACCAA- | |
| | | | CCGGCACT-(BHQ-1)***-GAAATAGG | |
| Internal control | 22 | C1038 | TCTCGAGCTCTGTACATGTCC | |
| plasmid | 23 | C1269 | TGAGGTAATTATAACCCGGGC | 252 |
| m13pSL3⁶ | | | | |

FAM is 6-carboxyfluorescein, a single isomer derivative of fluorescein
TET is tetrachlorofluorescein, a chemical relative of fluorescein
BHQ-1 and BHQ-2 are Black Hole QuencherTM dyes (Biosearch Technologies)
Cal Fluor® Red is a commercially available dye (Biosearch Technologies)
*na: not applicable
**^{L}N: locked nucleic acid (LNA) analog of a nucleotide
***This BHQ-1 moiety is covalently linked to the T nucleotide at position 30 of this oligonucleotide
¹Maheux A.F. et al., Water Res. 43: 3019-3028, 2009
²Frahm E. and Obst U., J. Microbiol. Meth. 52: 123-131, 2003.
³unpublished
⁴Guy R.A. et al., Appl. Environ. Microbiol. 69: 5178-5185, 2003
⁵Picard F.J. et al., J. Clin. Microbiol. 47: 751-757, 2009.
⁶Lansac N. et al., Eur. J. Clin. Microbiol. Infect. Dis. 19: 443-451, 2000.

TABLE 2
Real-time PCR amplification conditions

| Target organism | | |
|---|---|---|
| (target gene) | Denaturation | Amplification |
| E. coli (tuf) | 1 min @ 95° C. | 45 cycles of 2 sec @ 95° C., |
| | | 10 sec @ 58° C., 20 sec @ 72° C. |
| Enterococcus sp. | 3 min @ 95° C. | 45 cycles of 15 sec @ 95° C., |
| (16S rRNA) | | 60 sec @ 60° C. |
| E. faecalis-E. faecium | 1 min @ 95° C. | 45 cycles of 15 sec @ 95° C., |
| (mtlF-ddl) | | 10 sec @ 60° C., 20 sec @ 72° C. |
| C. parvum (COWP) | 3 min @ 95° C. | 45 cycles of 15 sec @ 95° C., |
| | | 60 sec @ 60° C. |
| G. intestinalis | 3 min @ 95° C. | 45 cycles of 15 sec @ 95° C., |
| (Giardin) | | 60 sec @ 60° C. |
| B.atrophaeus subsp. | 3 min @ 95° C. | 45 cycles of 15 sec @ 95° C., |
| globigii (atpD) | | 60 sec @ 60° C. |

Results and Discussion

This example demonstrates that the membrane dissolution procedure followed by a glass beads confinement allowed efficient recovery of E. coli and E. faecalis bacterial cells, and C. parvum and G. intestinalis (oo)cysts from cellulose ester filtration membranes in a state which is compatible with WGA and (rt)PCR amplification processes. The microbial nucleic acids subsequently extracted from the recovered microbial particles were then subjected to WGA-rtPCR amplification of specific genetic targets to achieve their detection.

Amplification products specific to E. coli and E. faecalis were respectively observed in samples containing as few as 1 cfu per 100 mL of water (see Table 3). This is a further demonstration of the applicability of the devised method for microbiological testing of potable water for which the presence of these bacteria is an indication of a potential risk of gastrointestinal disease(s). The glass beads-based cell lysis procedure serves to release DNA in a state that is compatible with nucleic acid detection technologies such as WGA and PCR.

Testing water for fecal contamination indicators is a major objective of public health and environmental regulatory authorities to insure the microbiological safety of water. The efficient recovery of bacteria cells (E. coli and E. faecalis) and their detection by molecular methods demonstrate the potential of the invention for the development of an integrated and rapid water diagnostic process designed to detect fecal contamination indicators and microbial pathogens, from a single water sample, and this, within one working day. Theoretically, this approach would provide a more rapid and specific response than currently approved methods.

The partial disintegration followed by the complete filtration membrane dissolution and glass beads confinement procedure allowed efficient recovery of Cryptosporidium oocysts and Giardia cysts from cellulose ester filtration membranes in a state compatible with WGA and PCR amplification processes. Amplification products specific to Cryptosporidium and Giardia were respectively observed in tested water samples spiked with microbes. As few as 2 Cryptosporidium oocysts and 1 Giardia cyst were detected when spiked in 100 mL of water (see Table 3). Only the internal control signals were observed in unspiked water samples.

As few as 60 B. atrophaeus subsp. globigii spores were also efficiently recovered and detected from spiked water samples. Serving as a cellular internal process control, this experiment shows that the DNA extracted from B. atrophaeus subsp. globigii spores is also suitable for the WGA-rtPCR amplification process.

This clearly demonstrates that microbial cells such as vegetative bacterial cells, parasite encysted forms and bacterial spores can be efficiently recovered by filtration on a mixed cellulose ester filtration membranes followed by the membrane dissolution/glass beads confinement procedure as revealed by the WGA-rtPCR method for nucleic acid amplification and specific detection. The combination of membrane dissolution, glass beads confinement and molecular detection procedures opens the possibility of simultaneous and/or retrospective detection of multiple waterborne microorganisms from a single water sample. This may be performed in the context of an integrated microbial surveillance system for the determination of water quality and safety. This demonstrates the versatility of the membrane dissolution/glass beads confinement procedure for the analysis of the microbiological quality of water, including fastidious and non-culturable microorganisms. The compatibility of the procedure with such a variety of microbial cells makes the present invention useful and valuable for rapid determination of the microbiological safety of water by non-culture based methods such as nucleic acid-based detection of microorganisms.

Finally, m13pSL3 was used as PCR amplification control aiming to determine the efficiency of the amplification process only. The presence of the m13pSL3-specific 252 by amplicon in all tests that are negative for the target organism demonstrates that chemical, molecular, or macromolecular components of the test do not significantly inhibit the PCR reaction and that PCR amplification was efficient.

TABLE 3
Presence/absence testing of microbial particles by culture-based
method and/or WGA-rtPCR amplification

| | | | Culture-based | WGA-rtPCR |
|---|---|---|---|---|
| | | | detection | detection |
| | | | Presence | Presence |
| | | | (+) or | (+) or |
| | | Average | absence (−) | absence (−) |
| Target | Experiment | bacterial count | for each | for each |
| organism | # | (cfu/100 mL) | replicate | replicate |
| E. coli | 1 | 10.3 ± 5.0 | +/+/+ | +/+/+ |
| | 2 | 5.3 ± 1.3 | +/+/+ | +/+/+ |
| | 3 | 3.3 ± 1.3 | +/+/+/+/+/+ | +/+/+/+/+/+ |
| | 4 | 1.5 ± 0.5 | +/+/+/+/+/+ | +/+/+/+/−/− |
| | 5 | 1.3 ± 0.9 | +/+/+/+/+/− | +/+/+/+/+/− |
| | 6 | 0.8 ± 0.4 | +/+/+/+/+/− | +/+/+/+/−/− |
| E. faecalis | 1 | 10.0 ± 1.6 | +/+/+ | +/+/+ |
| | 2 | 7.7 ± 1.3 | +/+/+ | +/+/+ |
| | 3 | 3.2 ± 1.2 | +/+/+/+/+/+ | +/+/+/+/−/− |
| | 4 | 2.0 ± 1.6 | +/+/+/+/+/− | +/+/+/−/−/− |
| | 5 | 1.2 ± 1.2 | +/+/+/−/−/− | +/+/+/+/−/− |
| | | | | WGA-rtPCR |
| | | Number of | | detection |
| | | spiked particles | | Presence |
| | | calculated from | | (+) or |
| | | Petroff-Hauser | | absence (−) |
| Target | Experiment | counts | | for each |
| organism | # | ([oo]cysts) | | replicate |
| C. parvum | 1 | 10 | na* | +/+/+/+/+/+ |
| | 2 | 5 | | +/+/+/−/−/− |
| | 3 | 5 | | +/+/+/−/−/− |
| | 4 | 2 | | +/−/−/−/−/− |
| | 5 | 1 | | −/−/−/−/−/− |
| G. intestinalis | 1 | 10 | na* | +/+/+/+/+/+ |
| | 2 | 5 | | +/+/+/+/+/+ |
| | 3 | 2 | | +/+/+/−/−/− |
| | 4 | 1 | | +/+/−/−/−/− |
| | 5 | 1 | | −/−/−/−/−/− |

(*na: not applicable)

### EXAMPLE 3

### Recovery of Escherichia coli, and Enterococcus faecalis, Cryptosporidium parvum Oocysts and Giardia intestinalis Cysts and Bacillus atrophaeus subsp. globigii Spores by Membrane Dissolution/Glass Beads Confinement Procedure and Detection by rtPCR

E. coli and enterococci are indicators of the fecal contamination of water. This example serves to demonstrate the efficiency of recovery of the procedure. Alternatively, this could also serve to develop a quantitative procedure for determining counts of microbial particles present in a sample.

Materials and Methods

Filtration membranes. Same as EXAMPLE 2.

Bacteria and methods used for culture-based testing of membrane-filtered samples. Same as EXAMPLE 2.

Cryptosporidium oocysts and Giardia cysts. Same as EXAMPLE 2.

Preparation of spiked water samples. Same as EXAMPLE 2, except that aliquots of bacterial cell dilutions and (oo)cyst preparations were spiked in spring water to produce suspensions targeting 400, 200, 80, 40, and 20 of each microbial particle type per 100 mL, assuming that a single rtPCR reaction would contain the equivalent of 10, 5, 2, 1, and 0.5 microbial particle(s). Molecular detection was achieved by the rtPCR procedure described below.

Membrane filtration of a water sample. Same as EXAMPLE 2.

Disintegration of the filtration membrane (primary dissolution). Same as EXAMPLE 2.

Complete dissolution of the filtration membrane (secondary dissolution). Same as EXAMPLE 2.

Rinsing of recovered microbial particles and removal of organic solvent(s). Same as EXAMPLE 2.

Lysis procedure for extraction of microbial nucleic acids. After centrifugation of the washed filtrate-glass beads suspension in the presence of TE buffer, the supernatant was removed using a micropipettor and discarded, with care taken to minimize glass bead agitation, leaving a residual volume of approximately 10 μL on top of the glass beads. Assuming that the dead volume of the glass beads is approx. 15 μL, 15 μL of TE buffer was added to the reaction mixture, bringing the total volume to approx. 40 μL, and the lysis of the cells contained in the pellet was achieved by vigorous mixing, at maximum speed, on a vortex mixer for 5 minutes at room temperature (23° C.). After a quick spin in a microcentrifuge, the reaction tube containing the cell lysate was incubated 2 minutes at 95° C., then briefly spun in a microcentrifuge, and kept at −20° C. until needed.

Real-time PCR (rtPCR) amplification. Same as EXAMPLE 2, except that 1 μL of each crude extract was transferred directly to the 24 μL rtPCR mixtures. Therefore, 1/40 of the membrane-trapped material is tested per each rtPCR reaction

Amplification of internal control (m13pSL3). Same as EXAMPLE 2.

Results and Discussion

This example demonstrates that the membrane dissolution procedure followed by a glass beads confinement allowed efficient recovery of E. coil and E. faecalis bacterial cells, and C. parvum and G. intestinalis (oo)cysts from cellulose ester filtration membranes in a state which is compatible with rtPCR amplification.

Amplification products specific to E. coli and E. faecalis were respectively observed in samples containing as few as 29 cfu per 100 mL of water (see Table 4). This suggests a very good efficiency of recovery since recovering as low as 29 microbial cells in a volume of 40 μL corresponds to delivering the genomic content of 0.73 cell in a single rtPCR reaction. This is a further demonstration of the applicability of the devised method for microbiological testing.

The filtration membrane dissolution followed by a glass beads confinement procedure allowed efficient recovery of Cryptosporidium oocysts and Giardia cysts from cellulose ester filtration membranes in a state compatible with rtPCR amplification. As few as 20 Cryptosporidium oocysts and 20 Giardia cysts were detected when spiked in 100 mL of water (see Table 4). This suggests a very good efficiency of recovery since recovering as low as 20 (oo)cysts in a volume of 40 μL corresponds to delivering the genomic content of 0.5 (oo)cyst in a single rtPCR reaction.

This clearly demonstrates that microbial cells such as vegetative bacterial cells, parasite encysted forms and bacterial spores can be efficiently recovered by filtration on a mixed cellulose ester filtration membranes followed by the application of the membrane dissolution/glass beads confinement procedure as revealed by the rtPCR method for nucleic acid amplification and specific detection. The combination of membrane dissolution, glass beads confinement and molecular detection procedures opens the possibility of simultaneous and/or retrospective detection of multiple waterborne microorganisms from a single water sample. This may be performed in the context of an integrated microbial surveillance system for the determination of water quality and safety. This demonstrates the versatility of the membrane dissolution/glass beads confinement procedure for the analysis of the microbiological quality of water, including fastidious and non-culturable microorganisms. The compatibility of the procedure with such a variety of microbial cells makes the present invention useful and valuable for rapid determination of the microbiological safety of water by non-culture based methods such as nucleic acid-based detection of microorganisms.

Finally, m13pSL3 was used as PCR amplification control aiming to determine the efficiency of the amplification process only. The presence of the m13pSL3-specific 252 by amplicon in all test negative for the target organism demonstrates that chemical, molecular, or macromolecular components of the test do not significantly inhibit the PCR reaction and that PCR amplification was efficient.

The invention has been described with certain exemplary embodiments. However, as obvious variations thereon will become apparent to a person skilled in the art, the invention is not to be considered as limited thereto.

TABLE 4
Detection of microbial particles by culture-based method
and/or rtPCR amplification

| | | Average | rtPCR detection |
|---|---|---|---|
| | | bacterial | Presence (+) or |
| Target | Experiment | count | absence (−) |
| organism | # | (cfu/100 mL) | for each replicate |
| E. coli | 1 | TNC* | +/+/+/+ |
| | 2 | TNC | +/+/+/+ |
| | 3 | 65.0 ± 5.5 | −/+/+ |
| | 4 | 63.0 ± 5.0 | +/+/+ |
| | 5 | 31.0 ± 2.2 | −/−/+ |
| | 6 | 29.0 ± 1.4 | −/+/+ |
| | 7 | 14.3 ± 1.7 | −/−/− |
| | 8 | 14.0 ± 0.8 | −/−/− |
| E. faecalis | 1 | TNC* | +/+/+/+ |
| | 2 | TNC | +/+/+/+ |
| | 3 | 77.7 ± 11.6 | +/+/+ |
| | 4 | 82.7 ± 7.1 | +/+/+ |
| | 5 | 30.7 ± 4.9 | +/+/+ |
| | 6 | 29.0 ± 3.3 | +/+/+ |
| | 7 | 14.7 ± 1.9 | +/−/− |
| | 8 | 13.3 ± 1.8 | +/−/+ |
| | | | rtPCR detection |
| | | Spiked number of particles | Presence (+) or |
| Target | Experiment | calculated from Petroff- | absence (−) |
| organism | # | Hauser counts ([oo]cysts) | for each replicate |
| C. parvum | 1 | 400 | +/+/+/+ |
| | 2 | 200 | +/+/+/+ |
| | 3 | 80 | +/+/+ |
| | 4 | 80 | +/−/+ |
| | 5 | 40 | +/+/+ |
| | 6 | 40 | +/+/− |
| | 7 | 20 | −/−/− |
| | 8 | 20 | +/−/− |
| G. intestinalis | 1 | 400 | +/+/+/+ |
| | 2 | 200 | +/+/+/+ |
| | 3 | 80 | +/+/+ |
| | 4 | 80 | +/+/+ |
| | 5 | 40 | +/+/+ |
| | 6 | 40 | +/+/+ |
| | 7 | 20 | +/+/+ |
| | 8 | 20 | +/−/+ |

*Too numerous to count
**na: not applicable

### EXAMPLE 4

### Performance of the Membrane Dissolution/Glass Beads Confinement Procedure in a Comparison Study of Culture-Based and Molecular Detection of E. coli and Enterococcus sp. in a Well Water Sample Spiked with Sewage

Sewage is a known natural source of waterborne microbial contaminants. This example serves to demonstrate that the membrane dissolution/glass beads confinement procedure can be used to efficiently recover and detect the contaminants contained in sewage, as compared with the culture-based methods for E. coli and enterococci.

Materials and Methods

Source of sewage and water. Sewage, obtained from the Water treatment plant of Saint-Nicolas (Québec, Canada) was spiked in a single well water sample collected from the Québec City area.

Filtration membranes. Same as EXAMPLE 2.

Preparation of spiked water samples. Aliquots of sewage were spiked in well water to produce suspensions targeting 100, 50, 10, 5, 1, 0.5, and 0.1 cfu per 100 mL. The number of spiked bacteria (cfu per 100 mL) was determined by plate count procedures on MI and mEI media. Molecular detection was achieved by the WGA-rtPCR procedure of EXAMPLE 2.

An internal process control, typically consisting of approximately 60 B. atropheus subsp. globigii spores was added to each spiked 100 mL water sample prior to filtration, as in EXAMPLE 2

Membrane filtration of a water sample. Same as EXAMPLE 2.

Disintegration of the filtration membrane (primary dissolution). Same as EXAMPLE 2.

Complete dissolution of the filtration membrane (secondary dissolution). Same as EXAMPLE 2.

Rinsing of recovered microbial particles and removal of organic solvent(s). Same as EXAMPLE 2.

Lysis procedure for extraction of microbial nucleic acids. Same as EXAMPLE 2.

Whole genome amplification (WGA) procedure. Same as EXAMPLE 2.

Real-time PCR (rtPCR) amplification of WGA-amplified nucleic acids. Same as EXAMPLE 2, except that for E. faecalis-E. faecium, 1 μL of the WGA reaction mixture was transferred directly to a 24 μL rtPCR mixture containing 50 mM KCl, 10 mM Tris-HCl (pH 9.1), 0.1% Triton X-100, 2.5 mM MgCl₂, 200 μM each deoxyribonucleoside triphosphate (dNTP; GE Healthcare, Baie d'Urfé, Québec, Canada), 3.3 μg/μL of bovine serum albumin (BSA; Sigma-Aldrich Canada Ltd., Oakville, Ontario, Canada), 0.025 enzyme unit (U) of Taq DNA polymerase (Promega, Madison, Wis., U.S.A.) combined to TaqStart antibody (Clontech, Palo Alto, Calif., U.S.A.). Independent rtPCR mixtures also contained 0.4 μM of each PCR amplification primer for E. faecalis-E. faecium (SEQ ID 7-10), and 0.2 μM of each dual-labeled (TaqMan) detection probe for E. faecalis-E. faecium (SEQ ID 11-12). The PCR mixtures were subjected to thermal cycling with a Rotor-Gene 3000 (Corbett Life Sciences, now QIAGEN Inc., Mississauga, Ontario, Canada) under the conditions presented in Table 2.

Amplification of internal control (m13pSL3). Same as EXAMPLE 2.

Results and Discussion

This example demonstrates that the membrane dissolution/glass beads confinement procedure allowed efficient recovery of E. coli and Enterococcus sp. bacterial cells contained in sewage (environmental bacteria). When compared to culture-based methods for their detection, the WGA-rtPCR was as efficient (see Table 5).

As few as 60 B. atrophaeus subsp. globigii spores were also efficiently recovered and detected from a well water sample spiked with sewage. Serving as a cellular internal process control, this experiment shows that the amplification of a B. atrophaeus subsp. globigii target is not inhibited by chemical/macromolecular components of the well water and of the sewage at the concentration used. This observation is confirmed by the positive amplification of the internal control m13pSL3.

TABLE 5
Detection of E. coli and Enterococcus sp. from well water spiked with
sewage

| Target | E. coli | E. coli | | | |
|---|---|---|---|---|---|
| bacterial | counts | detection | Enterococcus | Enterococcus | E. faecalis |
| count | on MI | by | sp. counts | sp. detection | detection |
| (cfu/ | (cfu/ | WGA- | on mEl | by WGA- | by WGA- |
| 100 mL) | 100 mL) | rtPCR | (cfu/100 mL) | rtPCR | rtPCR |
| 100 | 112 | + | 20 | + | + |
| 50 | 46 | + | 10 | + | + |
| 10 | 6 | + | 3 | + | − |
| 5 | 3 | + | 0 | + | − |
| 1 | 0 | − | 0 | + | − |
| 0.5 | 0 | − | 0 | + | − |
| 0.1 | 0 | − | 0 | + | − |
| unspiked | 0 | − | 0 | − | − |

The results of Table 5 show that a good correlation exists between E. coli counts on MI media and the detection by a specific E. coli rtPCR assay. The mEI medium has a specific pattern of detection of Enterococcus strains and hence, many environmental enterococcal isolates are not detected. The application of two rtPCR assays, a genus-specific assay and an assay targeting E. faecalis and E. faecium, might provide an indication of the contamination of both environmental and fecal origins.

### EXAMPLE 5

### Recovery of E. coli and Enterococcus sp. from Well Water Spiked with Sewage by the Membrane Dissolution/Glass Beads Confinement Procedure

Sewage is a known natural source of waterborne microbial contaminants. This example serves to demonstrate that the membrane dissolution/glass beads confinement procedure can be used to efficiently recover and detect the contaminants contained in sewage when spiked in different types of natural water samples.

Materials and Methods

Source of sewage and water samples. Sewage, obtained from the Water treatment plant of Saint-Nicolas (Québec, Canada) was spiked in well water samples (3 surface and 7 deep wells) from the Québec City area.

Filtration membranes. Same as EXAMPLE 2.

Cryptosporidium oocysts and Giardia cysts. Same as EXAMPLE 2. Spiking well water with (oo)cysts was done since preliminary experiments have shown that sewage was not contaminated with these microbial particles.

Preparation of spiked water samples. Aliquots of sewage were spiked in well water to produce suspensions targeting 100 E. coli cfu per 100 mL, 20 Enterococcus sp. cfu/mL, 100 Cryptosporidum and 100 Giardia (oo)cysts per 100 mL. Based on EXAMPLE 4, we have determined that sewage contains approx. 5 times less Enterococcus colony forming units than E. coli. The number of spiked bacteria (cfu per 100 mL) was determined by plate count procedures on MI and mEI media. Molecular detection was achieved by the WGA-rtPCR procedure of EXAMPLE 2.

An internal process control, typically consisting of approximately 60 B. atropheus subsp. globigii spores was added to each spiked 100 mL water sample prior to filtration, as in EXAMPLE 2.

Membrane filtration of a water sample. Same as EXAMPLE 2.

Disintegration of the filtration membrane (primary dissolution). Same as EXAMPLE 2.

Complete dissolution of the filtration membrane (secondary dissolution). Same as EXAMPLE 2.

Rinsing of recovered microbial particles and removal of organic solvent(s). Same as EXAMPLE 2.

Lysis procedure for extraction of microbial nucleic acids. Same as EXAMPLE 2.

Whole genome amplification (WGA) procedure. Same as EXAMPLE 2.

Real-time PCR (rtPCR) amplification of WGA-amplified nucleic acids. Same as EXAMPLE 2.

Amplification of internal control (m13pSL3). Same as EXAMPLE 2.

Results and Discussion

This example provides an indication of the robustness of the membrane dissolution/glass beads confinement procedure for the recovery and detection of many microbial particles from different types of natural well water samples (see Table 6). Not only WGA-rtPCR was positive with all samples for which culture-based evaluation was positive, but there is no evidence of significant rtPCR inhibition from the matrix, as observed with the amplification of the internal process and internal plasmid controls (not shown).

TABLE 6
Detection of microbial particles from well water samples spiked with
sewage and parasite (oo)cysts

| | | Pre- | | Microbial | | |
|---|---|---|---|---|---|---|
| | | spiking | Pre- | counts in | Spiked | WGA- |
| | | control | spiking | spiked | particle | rtPCR |
| | | counts | control | water | counts | in |
| | | (cfu/100 | WGA- | (cfu/100 | (particles/ | spiked |
| Sample | Target | mL) | rtPCR | mL) | 100 mL) | water |
| 1 | E. coli | 0 | — | 73 | na* | + |
| | Entero- | 0 | — | 16 | na* | + |
| | coccus sp. | | | | | |
| | Crypto- | na* | — | na* | (100)** | (+)** |
| | sporidium | | | | | |
| | Giardia | na* | — | na* | (100)** | (+)** |
| 2 | E. coli | 0 | — | TNC*** | na* | + |
| | Entero- | 0 | — | 12 | na* | + |
| | coccus sp. | | | | | |
| | Crypto- | na* | — | na* | (100)** | (+)** |
| | sporidium | | | | | |
| | Giardia | na* | — | na* | (100)** | (+)** |
| 3 | E. coli | 0 | — | TNC*** | na* | + |
| | Entero- | 0 | — | 22 | na* | + |
| | coccus sp. | | | | | |
| | Crypto- | na* | — | na* | (100)** | (+)** |
| | sporidium | | | | | |
| | Giardia | na* | — | na* | (100)** | (+)** |
| 4 | E. coli | 0 | — | 60 | na* | + |
| | Entero- | 0 | — | 16 | na* | + |
| | coccus sp. | | | | | |
| | Crypto- | na* | — | na* | (100)** | (+)** |
| | sporidium | | | | | |
| | Giardia | na* | — | na* | (100)** | (+)** |
| 5 | E. coli | 0 | — | TNC*** | na* | + |
| | Entero- | 0 | — | 17 | na* | + |
| | coccus sp. | | | | | |
| | Crypto- | na* | — | na* | (100)** | (+)** |
| | sporidium | | | | | |
| | Giardia | na* | — | na* | (100)** | (+)** |
| 6 | E. coli | 0 | — | 79 | na* | + |
| | Entero- | 0 | — | 12 | na* | + |
| | coccus sp. | | | | | |
| | Crypto- | na* | — | na* | (100)** | (+)** |
| | sporidium | | | | | |
| | Giardia | na* | — | na* | (100)** | (+)** |
| 7 | E. coli | 0 | — | 109 | na* | + |
| | Entero- | 0 | — | 10 | na* | + |
| | coccus sp. | | | | | |
| | Crypto- | na* | — | na* | (100)** | (+)** |
| | sporidium | | | | | |
| | Giardia | na* | — | na* | (100)** | (+)** |
| 8 | E. coli | 0 | — | 37 | 37 | + |
| | Entero- | 0 | — | 17 | 17 | + |
| | coccus sp. | | | | | |
| | Crypto- | na* | — | na* | (100)** | (+)** |
| | sporidium | | | | | |
| | Giardia | na* | — | na* | (100)** | (+)** |
| 9 | E. coli | 0 | — | TNC*** | na* | + |
| | Entero- | 0 | — | 16 | na* | + |
| | coccus sp. | | | | | |
| | Crypto- | na* | — | na* | (100)** | (+)** |
| | sporidium | | | | | |
| | Giardia | na* | — | na* | (100)** | (+)** |
| 10 | E. coli | 0 | — | TNC*** | na* | + |
| | Entero- | 0 | — | 10 | na* | + |
| | coccus sp. | | | | | |
| | Crypto- | na* | — | na* | (100)** | (+)** |
| | sporidium | | | | | |
| | Giardia | na* | — | na* | (100)** | (+)** |

*na: not applicable
**sewage tested negative for Cryptosporidium and Giardia. Water samples were spiked with 100 (oo)cysts. Results between brackets are for samples artificially spiked with (oo)cysts.
***Too numerous to count

### EXAMPLE 6

### Detection of Microbial Particles in Water from Agricultural Sites

Agricultural land runoff is a known source of microbial contamination of natural water reservoirs (lakes, rivers, streams, etc.). This example serves to demonstrate that the membrane dissolution/glass beads confinement procedure can be used to efficiently recover and detect B. atrophaeus subsp. globigii spores, incidentally used as internal process control, in natural water that is presumably contaminated by microorganisms and other organic matters such as humic acids. Preliminary experiments done with more than 200 samples have shown that B. atrophaeus subsp. globigii spores are not naturally detected in similar natural water samples.

Materials and Methods

Source of agricultural land water samples. Two 4 L samples collected from the Boyer Nord (near Saint-Anselme, Québec, Canada) and Bras d'Henri (near Saint-Narcisse-de-Beaurivage, Québec, Canada) rivers were analyzed.

Filtration membranes. Same as EXAMPLE 2.

Preparation of spiked water samples. Aliquots (10 and 100 mL) of river water and spring water (control) were each spiked with 1000 B. atrophaeus subsp. globigii spores. These samples were independently submitted to the membrane dissolution/glass beads confinement procedure to determine the level of molecular amplification inhibition attributable to molecular/macromolecular components such as humic acids, potentially present within the volume of filtered water. Molecular detection was achieved by the WGA-rtPCR procedure of EXAMPLE 2.

Membrane filtration of a water sample. Same as EXAMPLE 2.

Disintegration of the filtration membrane (primary dissolution). Same as EXAMPLE 2.

Complete dissolution of the filtration membrane (secondary dissolution). Same as EXAMPLE 2.

Rinsing of recovered microbial particles and removal of organic solvent(s). Same as EXAMPLE 2.

Lysis procedure for extraction of microbial nucleic acids. Same as EXAMPLE 2.

Whole genome amplification (WGA) procedure. Same as EXAMPLE 2.

Real-time PCR (rtPCR) amplification of WGA-amplified nucleic acids. Same as EXAMPLE 2.

Amplification of internal control (m13pSL3). Same as EXAMPLE 2.

Results and Discussion

This results shown in Table 7 demonstrate that although some unidentified molecular or macromolecular components of natural river water might exert some inhibition of the molecular amplification process, volumes of water of 10 and 100 mL are effectively testable by the membrane dissolution/glass beads confinement procedure, as demonstrated by the detection of B. atrophaeus subsp. globigii genetic material requires additional cycles of amplification to provide a positive signal when the enzymatic process is subjected to the influence of inhibitors.

However, the procedure was not modified to test these samples and the inclusion of procedures such as prefiltration, additional washing steps (for filters and for after secondary confinement) or inhibitor inactivation methods (metal chelation, thermal inactivation, etc.) could be incorporated to augment the efficiency of the overall procedure with larger volumes of natural water. In addition, nucleic acid purification prior to amplification may also help to remove inhibitors.

TABLE 7
Detection of B. atrophaeus subsp. globigii spores

| | Cycle threshold (CT) for detection of B. | |
|---|---|---|
| | atrophaeus subsp. globigii by WGA-rtPCR | |
| Sample | 10 mL | 100 mL |
| Control water | 37.0 | 38.5 |
| Boyer Nord River | 38.8 | 43.7 |
| Bras d'Henri River | 39.1 | 39.5 |

### EXAMPLE 7

### Comparative Evaluation of Four (4) Types of Filtration Membranes Made of Cellulose Esters for the Recovery of Microbial Particles by the Membrane Dissolution/Glass Beads Confinement Procedure

This example serves to demonstrate the performance of four widely used and commercially-available filtration membranes made of cellulose esters with the membrane dissolution/glass beads confinement procedure described herein, for the recovery of microbial particles and molecular detection by rtPCR and WGA-rtPCR.

Materials and Methods

Filtration membranes. The membrane dissolution followed by a glass beads confinement procedure was performed with membranes with a diameter of 47 mm and a porosity of 0.45 μm: plain and gridded sterile Metricel GN-6 mixed cellulose esters membrane filters (Pall Canada, Mississauga, ON, Canada), and plain and gridded sterile EZ-Pak™ mixed cellulose esters membrane filters (Millipore Canada, Mississauga, ON, Canada).

Preparation of spiked water samples. Spiked samples were prepared in commercially available spring water (Labrador, Ville d'Anjou, Québec, Canada). Cultures of E. coli or E. faecalis cells grown to logarithmic phase (0.5-0.6 OD_{600 nm}) were adjusted to a 0.5 McFarland standard, before being serially diluted ten-fold in PBS. Aliquots of the 10⁻⁵ dilution of E. coli and E. faecalis were used to prepare the spiked water samples. Particle counts provided by the supplier of Cryptosporidium oocysts and Giardia cysts were confirmed by counting with Petroff-Hauser chambers. Aliquots of both particle types were used to prepare spiked water samples also containing bacterial cells.

Aliquots of bacterial cell dilutions and (oo)cyst preparations were spiked in spring water to produce suspensions targeting 50 and 5 of each microbial particle type per 100 mL. The number of spiked bacteria (cfu per 100 mL) was estimated by plate count procedures, in multiple replicates on MI (E. coli), mEI (E. faecalis), or sheep blood agar (B. atrophaeus subsp. globigii; vegetative cells yield orange-colored colonies). Molecular detection was achieved by WGA-rtPCR (see EXAMPLE 2) and by rtPCR (see EXAMPLE 3).

An internal process control, typically consisting of approximately 60 B. atropheus subsp. globigii spores was added to each spiked 100 mL water sample prior to filtration. The methods and reagents for preparing B. atropheus subsp. globigii spores and detecting their nucleic acids is fully described elsewhere (International patent application number PCT/CA2003/01925). Molecular detection was achieved by WGA-rtPCR (see EXAMPLE 2) and by rtPCR (see EXAMPLE 3).

Membrane filtration of a water sample. Same as EXAMPLE 2.

Disintegration of the filtration membrane (primary dissolution). Same as EXAMPLE 2.

Complete dissolution of the filtration membrane (secondary dissolution). Same as EXAMPLE 2.

Rinsing of recovered microbial particles and removal of organic solvent(s). Same as EXAMPLE 2.

Lysis procedure for extraction of microbial nucleic acids. Same as EXAMPLE 2.

Whole genome amplification (WGA) procedure. Same as EXAMPLE 2.

Real-time PCR (rtPCR) amplification of WGA-amplified nucleic acids. Same as EXAMPLE 2.

Real-time PCR (rtPCR) amplification. Same as EXAMPLE 2, except that 1 μL of each crude extract was transferred directly to the 24 μL rtPCR mixtures.

Amplification of internal control (m13pSL3). Same as EXAMPLE 2.

Results and Discussion

These results shown in Table 8 demonstrate that, to some extent, all filtration membranes enable the recovery and molecular detection of the microbial targets that were spiked in the water samples using the membrane dissolution/confinement method described herein. A general observation is that GN-6 membranes seem to perform better than EZ-Pak membranes, as the treatment of the latter membranes might liberate chemical or macromolecular components that cause some molecular amplification inhibition, based on the increase of the number of cycles required for a positive signal or by the absence of amplification. Nucleic acid purification procedures, known by those skilled in the art, might alleviate PCR inhibition. However, the sample size is too limited to derive statistically valid correlations.

On the other hand, since the four membranes types have only been evaluated by the efficiency of molecular amplification, it must be understood that other modes of detection such as microscopy or antibody-based methods might provide different observations.

TABLE 8
Comparison of four commercially-available filtration membranes made of
cellulose esters for the recovery and detection of E. coli, E. faecalis, C. parvum, G.
intestinalis, and B. atrophaeus subsp. globigii by rtPCR alone and with prior
molecular amplification by WGA.

| | | | Average | GN-6 | | GN-6 | | EZ-Pak | | EZ-Pak | |
|---|---|---|---|---|---|---|---|---|---|---|---|
| | | | bacterial | plain | | gridded | | plain | | gridded | |
| Target | Spiked | | count | WGA⁻ | WGA⁺ | WGA⁻ | WGA⁺ | WGA⁻ | WGA⁺ | WGA⁻ | WGA⁺ |
| organism | particles | | (cfu/100 mL) | CT* | CT* | CT* | CT* | CT* | CT* | CT* | CT* |
| E. coli | 50 | | 30.0 | — | 34.5 | — | 38.0 | — | 39.1 | — | 37.8 |
| | 5 | | 7.5 | nd | — | nd | — | nd | — | nd | — |
| E. faecalis | 50 | | 26.5 | 34.9 | 29.1 | — | 32.3 | — | 31.0 | 38.0 | 32.3 |
| | 5 | | 7.0 | nd | 36.3 | nd | 38.7 | nd | — | nd | — |
| C. parvum | 50 | | na | — | 28.6 | — | 29.6 | — | 32.2 | — | 33.8 |
| | 5 | | na | nd | 28.4 | nd | 29.1 | nd | 33.2 | nd | — |
| G. intestinalis | 50 | | na | 39.1 | 31.4 | 39.5 | 28.5 | 40.3 | 30.9 | 40.2 | 30.4 |
| | 5 | | na | nd | — | nd | 33.6 | nd | — | nd | 34.4 |
| B. atrophaeus | 60 | (50) | 46.0** | — | 32.4 | — | 32.6 | — | — | — | — |
| subsp. globigii | 60 | (5) | 26.0** | — | 34.0 | — | 30.1 | — | — | — | — |

*CT: cycle threshold
**For B. atrophaeus subsp. globigii, the average count represents the number of colonies derived from the spiked spores, enumerated by microscopic evaluation.
na: not applicable,
nd: not determined

### EXAMPLE 8

### Determination of the Efficiency of the Membrane Dissolution/Glass Beads Confinement Procedure with a Sample Volume of 1000 mL

This example serves to demonstrate the membrane dissolution/glass beads confinement procedure, primarily used for the analysis of 100 mL water samples can also be used to efficiently analyze water samples of 1000 mL to recover and detect, for example, microbial pathogens different from fecal contamination indicators.

Materials and Methods

Filtration membranes. Same as EXAMPLE 2.

Preparation of spiked water samples. Same as EXAMPLE 2.

Aliquots of bacterial cell dilutions and (oo)cyst preparations were spiked in spring water to produce suspensions targeting 50 and 5 of each microbial particle type per 100 mL, and 50 and 5 of each microbial particle type per 1000 mL. The number of spiked bacteria (cfu per 100 mL or cfu per 1000 mL) was estimated by plate count procedures, in multiple replicates on MI (E. coli), mEI (E. faecalis), or sheep blood agar (B. atrophaeus subsp. globigii; vegetative cells yield orange-colored colonies). Molecular detection was achieved by WGA-rtPCR (see EXAMPLE 2) and by rtPCR (see EXAMPLE 3).

An internal process control, typically consisting of approximately 60 B. atropheus subsp. globigii spores was added to each spiked 100 or 1000 mL water sample prior to filtration. The methods and reagents for preparing B. atropheus subsp. globigii spores and detecting their nucleic acids is fully described elsewhere (International patent application number PCT/CA2003/01925). Molecular detection was achieved by WGA-rtPCR (see EXAMPLE 2) and by rtPCR (see EXAMPLE 3).

Membrane filtration of a water sample. Same as EXAMPLE 2.

Disintegration of the filtration membrane (primary dissolution). Same as EXAMPLE 2.

Complete dissolution of the filtration membrane (secondary dissolution). Same as EXAMPLE 2.

Rinsing of recovered microbial particles and removal of organic solvent(s). Same as EXAMPLE 2.

Lysis procedure for extraction of microbial nucleic acids. Same as EXAMPLE 2.

Whole genome amplification (WGA) procedure. Same as EXAMPLE 2.

Real-time PCR (rtPCR) amplification of WGA-amplified nucleic acids. Same as EXAMPLE 2.

Real-time PCR (rtPCR) amplification. Same as EXAMPLE 7.

Amplification of internal control (m13pSL3). Same as EXAMPLE 2.

Results and Discussion

This result shown in Table 9 demonstrates that the membrane dissolution/bead confinement procedure can be used to efficiently analyze finished water samples for their microbial particle content.

Assuming that 1000 mL of finished water contains limited amounts of molecular amplification (WGA and/or PCR) inhibitors, based on a slight increase of cycle threshold results, this suggests that sample volumes between 100-1000 mL can be efficiently treated by the membrane dissolution/bead confinement procedure, but that 1000 mL is certainly not the superior limit of sample volume. The superior sample volume limit would therefore depend on the concentration of molecular amplification inhibitors and the nature of the microbial contaminants.

Depending on the water type (spring water, agricultural water, seawater, etc.), it is believed that additional treatments of the filter or of the recovered particles (microbial or else) might be included to remove inhibitors and alleviate molecular amplification inhibition. On the other hand, we demonstrate that microbial particles are efficiently recovered, but for methods of detection differing from molecular amplification, these limitations might not apply.

TABLE 9
Evaluation of the membrane dissolution/bead confinement procedure for
recovery and detection of E. coli, E. faecalis, C. parvum, G. intestinalis, and B.
atrophaeus subsp. globigii by rtPCR alone and with prior molecular amplification
by WGA, from 100 and 1000 mL spiked water samples.

| | | | 100 mL water samples | | | 1000 mL water samples | | |
|---|---|---|---|---|---|---|---|---|
| | | | Average | | | Average | | |
| Target | Spiked | | bacterial count | WGA⁻ | WGA⁺ | bacterial count | WGA⁻ | WGA⁺ |
| organism | particles | | (cfu/100 mL) | CT* | CT* | (cfu/1000 mL) | CT* | CT* |
| E. coli | 50 | | 69.0 | 36.4 | 28.5 | 74.0 | 36.4 | 29.8 |
| | 5 | | 4.0 | nd | 35.6 | 7.0 | nd | 35.6 |
| E. faecalis | 50 | | 28.0 | 35.4 | 28.8 | 38.0 | 35.0 | 29.3 |
| | 5 | | 7.0 | nd | 43.1 | 5.0 | nd | 30.3 |
| C. parvum | 50 | | na | 36.1 | 27.6 | na | 37.1 | 30.4 |
| | 5 | | na | nd | 31.2 | na | nd | 30.8 |
| G. intestinalis | 50 | | na | 37.8 | 29.7 | na | 35.3 | 31.2 |
| | 5 | | na | nd | 35.9 | na | nd | 32.2 |
| B. atrophaeus | 60 | (50) | 18.0** | — | 34.2 | 18.0** | — | — |
| subsp. globigii | 60 | (5) | 26.0** | — | 34.3 | 22.0** | — | 35.8 |

*CT: cycle threshold
**For B. atrophaeus subsp. globigii, the average count represents the number of colonies derived from the spiked spores, enumerated by microscopic evaluation.
na: not applicable,
nd: not determined

The invention has been described with certain exemplary embodiments. However, as obvious variations thereon will become apparent to a person skilled in the art, the invention is not to be considered as limited thereto.

1. A method for the recovery of cells or microorganisms from a cellulose ester filtration matrix comprising or suspected of comprising the cells or microorganisms, the method comprising sequentially:
a. Obtaining a partially disintegrated filtration matrix from the cellulose ester filtration matrix by partial dissolution of the filtration matrix in a first organic solvent and sedimentation, and;
b. Treating the partially disintegrated filtration matrix by replacing the first organic solvent with a second organic solvent allowing complete dissolution of the partially disintegrated filtration matrix.

2. (canceled)

3. The method of claim 1, wherein the first organic solvent is methanol.

4-5. (canceled)

6. The method of claim 3, wherein the second organic solvent is acetone.

7. (canceled)

8. The method of claim 1, further comprising confining the cells or microorganisms in a mobile or static confinement structure or matrix.

9. The method of claim 8, wherein confinement is performed by centrifugation with or over beads.

10. The method of claim 8, wherein the second organic solvent is replaced with a buffer suitable for a detection method.

11. The method of claim 1, wherein the cells are selected from the group consisting of bacterial, archaeal, or eucaryal cells and wherein the microorganisms are selected from the group consisting of bacterium, bacterial spores, archaea, protozoans, protozoan cysts, multicellular parasites, fungi, fungal spores, viruses and bacteriophages.

12. (canceled)

13. The method of claim 11, wherein the bacteria is Bacillus atrophaeus subsp. globigii or a spore thereof.

14. The method of claim 11, wherein the bacterium are fecal contamination indicators or spores thereof and wherein the protozoan or protozoan (oo)cyst is selected from the group consisting of Cryptosporidium parvum, C. hominis, Giardia intestinalis and (oo)cysts thereof.

15. The method of claim 14, wherein the fecal contamination indicators are selected from the group consisting of E. coli, Enterococcus sp, E. faecium and E. faecalis or spores thereof.

16. (canceled)

17. The method of claim 1, wherein the microorganisms are recovered from a gaseous matrix, liquid matrix, potable water, natural water reservoirs, sewage, well water or water from treatment plan.

18. (canceled)

19. The method of claim 17, wherein the method allows for the recovery of at least 1 cfu or 1 microorganism per tested volume.

20-26. (canceled)

27. The method of claim 1, further comprising detecting the cells or microorganisms.

28. The method of claim 27, wherein detection of multiple microorganism species is performed.

29. A kit for the recovery of cells or microorganisms from a cellulose ester filtration matrix comprising or suspected of comprising cells or microorganisms, the kit comprising: a first vial comprising a first organic solvent capable of partially disintegrating the filtration matrix, a second vial comprising a second organic solvent capable of completely dissolving the filtration matrix and optionally comprising instructions for the sequential partial disintegration of the filtration matrix and complete dissolution of the filtration matrix.

30. The kit of claim 29, further including a vial comprising a mobile confinement structure or matrix.

31. (canceled)

32. The kit of claim 29, wherein the first organic solvent is methanol.

33. The kit of claim 32, wherein the second organic solvent is acetone.

34. The kit of claim 30, wherein the mobile confinement structure or matrix comprises beads.

35. (canceled)

36. The kit of claim 29, further comprising reagents for the specific detection of cells or microorganisms by nucleic acid amplification.

37. (canceled)
